SEQUENCE LISTING (25 sequences)

SEQ ID NO: 1 - DNA, 51 bp, synthetic construct
  misc_feature 1..51: note = Synthetic: RLuc8.6 F
  source 1..51: mol_type = other DNA
tgtacgacgatgacgataaggatccgatggcttccaaggtgtacgaccccg

SEQ ID NO: 2 - DNA, 51 bp, synthetic construct
  misc_feature 1..51: note = Synthetic: RLuc8.6 R
  source 1..51: mol_type = other DNA
agcagccggatcaagcttcgaattcttactgctcgttcttcagcacgcgct

SEQ ID NO: 3 - DNA, 51 bp, synthetic construct; identical to SEQ ID NO: 1
  misc_feature 1..51: note = Synthetic: RLuc8.6 F
  source 1..51: mol_type = other DNA

SEQ ID NO: 4 - DNA, 23 bp, synthetic construct
  misc_feature 1..23: note = Synthetic: RLuc8.6 R
  source 1..23: mol_type = other DNA
ctgctcgttcttcagcacgcgct

SEQ ID NO: 5 - DNA, 120 bp, synthetic construct
  misc_feature 1..120: note = Synthetic: DNA fragment of IKVAV
  source 1..120: mol_type = other DNA
gagcgcgtgctgaagaacgagcagggaggaagtggcagctctggcggcagtggagggtctggtggcagcggaatcaaagtcgccgtgtaagaattcgaagcttgatccggctgctaacaa

SEQ ID NO: 6 - DNA, 51 bp, synthetic construct; identical to SEQ ID NO: 1
  misc_feature 1..51: note = Synthetic: RLuc8.6 F
  source 1..51: mol_type = other DNA

SEQ ID NO: 7 - DNA, 23 bp, synthetic construct
  misc_feature 1..23: note = Synthetic: RLuc8.6 R
  source 1..23: mol_type = other DNA
ctgctcgttcttcagcacgcgct

SEQ ID NO: 8 - DNA, 120 bp, synthetic construct
  misc_feature 1..120: note = Synthetic: DNA fragment of YIGSR
  source 1..120: mol_type = other DNA
gagcgcgtgctgaagaacgagcagggaggaagtggcagctctggcggcagtggagggtctggtggcagcggatacattggttccagataagaattcgaagcttgatccggctgctaacaa

SEQ ID NO: 9 - DNA, 51 bp, synthetic construct; identical to SEQ ID NO: 1
  misc_feature 1..51: note = Synthetic: RLuc8.6 F
  source 1..51: mol_type = other DNA

SEQ ID NO: 10 - DNA, 23 bp, synthetic construct
  misc_feature 1..23: note = Synthetic: RLuc8.6 R
  source 1..23: mol_type = other DNA
ctgctcgttcttcagcacgcgct

SEQ ID NO: 11 - DNA, 120 bp, synthetic construct
  misc_feature 1..120: note = Synthetic: DNA fragment of GRGDS
  source 1..120: mol_type = other DNA
gagcgcgtgctgaagaacgagcagggaggaagtggcagctctggcggcagtggagggtctggtggcagcggaggtcgtggtgatagttaagaattcgaagcttgatccggctgctaacaa

SEQ ID NO: 12 - DNA, 57 bp, synthetic construct
  misc_feature 1..57: note = Synthetic: AADC-IKVAV F
  source 1..57: mol_type = other DNA
tgtacgacgatgacgataaggatccgatgaacgcaagtgaattccgaaggagaggga

SEQ ID NO: 13 - DNA, 53 bp, synthetic construct
  misc_feature 1..53: note = Synthetic: AADC-IKVAV R
  source 1..53: mol_type = other DNA
agcagccggatcaagcttcgaattcttacacggcgactttgattccgctgcca

SEQ ID NO: 14 - DNA, 56 bp, synthetic construct
  misc_feature 1..56: note = Synthetic: AADC F
  source 1..56: mol_type = other DNA
tgacgataaggatcgatggggatccatgaacgcaagtgaattccgaaggagaggga

SEQ ID NO: 15 - DNA, 22 bp, synthetic construct
  misc_feature 1..22: note = Synthetic: AADC R
  source 1..22: mol_type = other DNA
ctccctctctgctcgcagcacg

SEQ ID NO: 16 - DNA, 120 bp, synthetic construct
  misc_feature 1..120: note = Synthetic: DNA fragment of IKVAV
  source 1..120: mol_type = other DNA
acgtgctgcgagcagagagggagggaggaagtggcagctctggcggcagtggagggtctggtggcagcggaatcaaagtcgccgtgtagggtaccatatgggaattcgaagcttggctgt

SEQ ID NO: 17 - DNA, 1032 bp, synthetic construct
  misc_feature 1..1032: note = Synthetic: RLuc8.6
  source 1..1032: mol_type = other DNA
atgcggggttctcatcatcatcatcatcatggtatggctagcatgactggtggacagcaaatgggtcgggatctgtacgacgatgacgataaggatccgatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctacctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactgggggagcgctctggcctttcactacgcctacgagcaccaagacaggatcaaggccatcgtccatatggagagtgtcgtggacgtgatcgagtcctggatggggtggcctgacatcgaggaggagctggccctgatcaagagcgaagagggcgagaaaatggtgcttgagaataacttcttcgtcgagaccctgttgccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagctgttcatcgagtccgaccctgggttcttttccaacgctattgtcgagggagctaagaagttccctaacaccgagttcgtgaaggtgaagggcctccacttcctccaggaggacgctccagatgaaatgggtaagtacatcaagagcttcgtggagcgcgtgctgaagaacgagcag

SEQ ID NO: 18 - DNA, 1095 bp, synthetic construct
  misc_feature 1..1095: note = Synthetic: RLuc8.6-IKVAV
  source 1..1095: mol_type = other DNA
atgcggggttctcatcatcatcatcatcatggtatggctagcatgactggtggacagcaaatgggtcgggatctgtacgacgatgacgataaggatccgatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctacctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactgggggagcgctctggcctttcactacgcctacgagcaccaagacaggatcaaggccatcgtccatatggagagtgtcgtggacgtgatcgagtcctggatggggtggcctgacatcgaggaggagctggccctgatcaagagcgaagagggcgagaaaatggtgcttgagaataacttcttcgtcgagaccctgttgccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagctgttcatcgagtccgaccctgggttcttttccaacgctattgtcgagggagctaagaagttccctaacaccgagttcgtgaaggtgaagggcctccacttcctccaggaggacgctccagatgaaatgggtaagtacatcaagagcttcgtggagcgcgtgctgaagaacgagcagggaggaagtggcagctctggcggcagtggagggtctggtggcagcggaatcaaagtcgccgtg

SEQ ID NO: 19 - DNA, 1095 bp, synthetic construct
  misc_feature 1..1095: note = Synthetic: RLuc8.6-YIGSR
  source 1..1095: mol_type = other DNA
atgcggggttctcatcatcatcatcatcatggtatggctagcatgactggtggacagcaaatgggtcgggatctgtacgacgatgacgataaggatccgatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctacctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactgggggagcgctctggcctttcactacgcctacgagcaccaagacaggatcaaggccatcgtccatatggagagtgtcgtggacgtgatcgagtcctggatggggtggcctgacatcgaggaggagctggccctgatcaagagcgaagagggcgagaaaatggtgcttgagaataacttcttcgtcgagaccctgttgccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagctgttcatcgagtccgaccctgggttcttttccaacgctattgtcgagggagctaagaagttccctaacaccgagttcgtgaaggtgaagggcctccacttcctccaggaggacgctccagatgaaatgggtaagtacatcaagagcttcgtggagcgcgtgctgaagaacgagcagggaggaagtggcagctctggcggcagtggagggtctggtggcagcggatacattggttccaga

SEQ ID NO: 20 - DNA, 1095 bp, synthetic construct
  misc_feature 1..1095: note = Synthetic: RLuc8.6-GRGDS
  source 1..1095: mol_type = other DNA
atgcggggttctcatcatcatcatcatcatggtatggctagcatgactggtggacagcaaatgggtcgggatctgtacgacgatgacgataaggatccgatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctacctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactgggggagcgctctggcctttcactacgcctacgagcaccaagacaggatcaaggccatcgtccatatggagagtgtcgtggacgtgatcgagtcctggatggggtggcctgacatcgaggaggagctggccctgatcaagagcgaagagggcgagaaaatggtgcttgagaataacttcttcgtcgagaccctgttgccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagctgttcatcgagtccgaccctgggttcttttccaacgctattgtcgagggagctaagaagttccctaacaccgagttcgtgaaggtgaagggcctccacttcctccaggaggacgctccagatgaaatgggtaagtacatcaagagcttcgtggagcgcgtgctgaagaacgagcagggaggaagtggcagctctggcggcagtggagggtctggtggcagcggaggtcgtggtgatagt

SEQ ID NO: 21 - DNA, 1602 bp, synthetic construct
  misc_feature 1..1602: note = Synthetic: AADC-IKVAV
  source 1..1602: mol_type = other DNA
atgcggggttctcatcatcatcatcatcatggtatggctagcatgactggtggacagcaaatgggtcgggatctgtacgacgatgacgataaggatccgatgaacgcaagtgaattccgaaggagagggaaggagatggtggattacatggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggtgacgcactggcacagcccctacttcttcgcctacttccccactgccagctcgtacccggccatgcttgcggacatgctgtgcggggccattggctgcatcggcttctcctgggcggcaagcccagcatgcacagagctggagactgtgatgatggactggctcgggaagatgctggaactaccaaaggcatttttgaatgagaaagctggagaagggggaggagtgatccagggaagtgccagtgaagccaccctggtggccctgctggccgctcggaccaaagtgatccatcggctgcaggcagcgtccccagagctcacacaggccgctatcatggagaagctggtggcttactcatccgatcaggcacactcctcagtggaaagagctgggttaattggtggagtgaaattaaaagccatcccctcagatggcaacttcgccatgcgtgcgtctgccctgcaggaagccctggagagagacaaagcggctggcctgattcctttctttatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggatccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggagggaggaagtggcagctctggcggcagtggagggtctggtggcagcggaatcaaagtcgccgtg

SEQ ID NO: 22 - DNA, 4768 bp, synthetic construct
  misc_feature 1..4768: note = Synthetic: pRSETb vector
  source 1..4768: mol_type = other DNA
ttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggatctcgatcccgcgaaattaatacgactcactatagggagaccacaacggtttccctctagaaataattttgtttaactttaagaaggagatatacatatgcggggttctcatcatcatcatcatcatggtatggctagcatgactggtggacagcaaatgggtcgggatctgtacgacgatgacgataaggatccgatggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttccggctggaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaagaattcgaagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaac

SEQ ID NO: 23 - protein, 5 aa, synthetic construct
  REGION 1..5: note = Synthetic: peptide
IKVAV

SEQ ID NO: 24 - protein, 5 aa, synthetic construct
  REGION 1..5: note = Synthetic: peptide
GRGDS

SEQ ID NO: 25 - protein, 5 aa, synthetic construct
  REGION 1..5: note = Synthetic: peptide
YIGSR

(19) United States
(12) Patent Application Publication
(10) Pub. No.: US 2025/0195629 A1
(43) Pub. Date: Jun. 19, 2025

(54) SITE-SPECIFIC BRAIN THERAPEUTICS

(71) Applicant: William Marsh Rice University, Houston, TX (US)

(72) Inventors: Jerzy Olgierd Szablowski, Houston, TX (US); Zhimin Huang, Houston, TX (US)

(21) Appl. No.: 18/974,314

(22) Filed: Dec. 9, 2024

Related U.S. Application Data
(60) Provisional application No. 63/607,829, filed on Dec. 8, 2023.

(51) Int. Cl. A61K 38/51; A61K 31/137; C12N 9/88

(52) U.S. Cl. CPC A61K 38/51; A61K 31/137; C12N 9/88; C12Y 401/01028

(57) ABSTRACT

The present disclosure relates to an approach, referred to as Regionally Activated Interstitial Drugs (RAID), that does not require viral vectors, but allows for tunable, noninvasive, long-term neuromodulation with small molecules. RAID utilizes noninvasive delivery of an engineered protein enzyme into the brain, which then binds to the brain parenchyma and can locally convert a blood-brain-barrier (BBB)-permeable inert prodrug into an active neuromodulatory drug. As long as the RAID enzyme is present in the parenchyma, localized neuromodulation can be achieved with systemic administration of the BBB-permeable prodrug even in the absence of the opened BBB. Alternatively, gene delivery encoding the RAID enzyme offers prolonged expression and precise spatial control, enabling long-term and adaptable neuromodulation.

### REFERENCE TO ELECTRONIC SEQUENCE LISTING

The application contains a Sequence Listing which has been submitted electronically in .XML format and is hereby incorporated by reference in its entirety. Said .XML copy, created on Feb. 21, 2025, is named “RICE0026.xml” and is 43,867 bytes in size. The sequence listing contained in this .XML file is part of the specification and is hereby incorporated by reference herein in its entirety.

### CROSS-REFERENCE TO RELATED APPLICATIONS

This application claims priority to and the benefit of U.S. Provisional Application No. 63/607,829, entitled “SITE-SPECIFIC BRAIN THERAPEUTICS,” filed Dec. 8, 2023, which is herein incorporated by reference in its entirety for all purposes.

### TECHNICAL FIELD

The subject matter disclosed herein relates to modulating neuronal activity noninvasively at specific brain regions for extended periods of time. In particular, the combination of focused ultrasound, engineered enzymes, and prodrugs enabled small molecule therapeutics to exert their action in specific brain regions for multiple days.

### BACKGROUND

Many brain therapies, such as surgical resections or gene therapy, are invasive, risky, and irreversible, but their outcome is uncertain.

Physiologic brain activity and pathogenesis of brain disorders depends on neuronal activity in specific brain regions. Modulating neuronal activity noninvasively at specific brain regions for extended periods of time would allow new studies of brain circuitry and provide new therapeutic options with potentially fewer nonspecific effects. Unfortunately, neuromodulation with spatiotemporal precision over long periods of time is a major challenge. On one hand, therapeutics are non-invasive, and socially acceptable tools to control neurons, but they diffuse throughout the brain acting on multiple brain regions with no spatial specificity. Recent studies have shown that spatially precise control of neuronal activity can be achieved with a combination of focused ultrasound blood-brain barrier opening (FUS-BBBO) and delivery of small molecule drugs or gene therapy carrying chemogenetic receptors, in an approach termed Acoustically Targeted Chemogenetics (ATAC). However, delivery of small molecule therapeutics is short-lived, and their activity is confounded by presence of an opened BBB. On the other hand, chemogenetic approaches such as ATAC allow for long-term activity, but can only be feasibly administered once due to the development of immune response against the vectors, and are hampered by potential risks of toxicity, immunogenicity of the viral vectors.

This invention was funded in part by the Robert A. Welch Foundation under Welch Grant No. C-2048.

### BRIEF DESCRIPTION

Many brain therapies, such as surgical resections or gene therapy, are invasive, risky, and irreversible, but their outcome is uncertain. Disclosed herein are embodiments directed towards noninvasively testing invasive therapies. Accordingly, embodiments herein are directed towards Regionally Activated Interstitial Drugs (RAID). RAID provides noninvasive, multi-day, pharmacological control over specific sites of an intact brain. Embodiments herein disclose use of focused ultrasound techniques to noninvasively deliver engineered protein enzymes to one or more specific brain regions. Within the specific brain regions, one or more enzymes may bind to the brain parenchyma for one or more days and may convert inert BBB-permeable prodrugs into active drugs. RAID control neuronal function and behavior for several days, mimicking the effects of invasive drug delivery. As such, use of RAID offers versatility and can be applied to various enzymes and prodrug pair to control various aspects of central nervous system physiology.

In one embodiment, a method is provided for applying site-specific brain therapeutics. In accordance with this embodiment, an enzyme is delivered to a selected site of a brain. An inactive prodrug is converted, via the enzyme, into an active drug at the selected site of the brain.

In accordance with another embodiment, an engineered enzyme is provided. In accordance with this embodiment, the engineered enzyme comprises: an aromatic-L-amino-acid decarboxylase (AADC) and an extracellular matrix (ECM)-mimicking peptide fused to the AADC.

In accordance with a further embodiment, a method is provided for modulating neuronal activity at a spatially specific site within a brain. In accordance with this embodiment, an inert prodrug is administered to a subject. The inert prodrug is capable of penetrating an intact blood-brain-barrier and the inert prodrug is converted to an active drug at the spatially specific site within the brain. A dose of the inert prodrug varies over time to achieve a corresponding or modulated dose of the active drug at the spatially specific site.

### BRIEF DESCRIPTION OF THE DRAWINGS

In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings, in which:

FIG. 1a illustrates an embodiment of Regionally Activated Interstitial Drugs (RAID) directed to site-specific non-genetic therapeutics including an enzyme delivered to a selected site of brain specifically converts an inactive but BBB permeable prodrug (e.g., P) into a neuroactive drug (e.g., D), in accordance with embodiments described herein;

FIG. 1b illustrates an embodiment of RAID in which one or more catalytic enzymes are delivered to the brain via FUS-BBBO, wherein the catalytic enzymes bind within the brain interstitium to increase a residence time of the catalytic enzymes), in accordance with embodiments described herein;

FIG. 2a illustrates an embodiment of noninvasively delivered enzyme retains activity in the brain interstitium including delivery of a recombinant luciferase (e.g., RLuc8.6) to a mouse brain through FUS-BBBO and conducted bioluminescent imaging to assess the efficacy of targeted enzyme delivery and its activity, in accordance with embodiments described herein;

FIG. 2b illustrates representative immunostaining images for RLuc8.6 in a mouse 1 h after FUS-BBBO (targeting 4 sites in left striatum) and systemic administration of RLuc8.6 (150 mg/kg) including a magnified view of the FUS-targeted region (shown on the left), and the contralateral site (shown on the right), in accordance with embodiments described herein;

FIG. 2c illustrates an embodiment directed to quantification of average fluorescence intensity of RLuc8.6 over the FUS-targeted region and the contralateral site, in accordance with embodiments described herein;

FIG. 2d illustrates an embodiment directed to in vivo bioluminescent imaging representative data at field of view A (3.9 cm across), wherein FUS targeting one site at left striatum was followed by an IV injection of RLuc8.6 (8 mg/kg) or PBS buffer control, in accordance with embodiments described herein;

FIG. 2e illustrates an embodiment directed to quantification of bioluminescence average radiance (p/s/cm²/sr) at field of view C (13 cm across) in the head region 1 h after FUS-BBBO, in accordance with embodiments described herein;

FIG. 3a illustrates representative immunostaining images after RLuc8.6 delivery related to FIG. 2b including fluorescence imaging of coronal section with RLuc8.6 retention, in accordance with embodiments of the present disclosure;

FIG. 3b illustrates the fluorescence imaging of coronal section of FIG. 4b at higher magnifications, in accordance with embodiments of the present disclosure;

FIG. 4a illustrates representative images and data of in vivo BLI at different time points after delivering RLuc8.6, in accordance with embodiments of the present disclosure;

FIG. 4b illustrates a graph directed to quantification of bioluminescence average radiance (p/s/cm2/sr) at field of view C (13 cm) in the head region 24 h after FUS-BBBO, in accordance with embodiments of the present disclosure;

FIG. 4c illustrates a graph directed to quantification of bioluminescence average radiance (p/s/cm2/sr) at field of view C (13 cm) in the head region 48 h after FUS-BBBO, in accordance with embodiments of the present disclosure;

FIG. 4d illustrates a graph directed to quantification of bioluminescence average radiance (p/s/cm2/sr) at field of view C (13 cm) in the head region 96 h after FUS-BBBO, in accordance with embodiments of the present disclosure;

FIG. 5a illustrates an embodiment directed towards engineering an enzyme to improve retention in a brain including a schematic diagram of engineered cell-adhesive peptides that mimic the ECM for attaching prodrug enzymes to brain cells, in accordance with embodiments described herein;

FIG. 5b illustrates an embodiment of an experimental scheme for measuring retention of unmodified or engineered RLuc8.6 in a mouse brain, in accordance with embodiments described herein;

FIG. 5c illustrates ex vivo bioluminescent imaging representative data for mice at 2 days after FUS-BBBO, in accordance with embodiments described herein;

FIG. 5d illustrates a graph including a sum of bioluminescence average radiance (p/s/cm²/sr) in five sections of each mouse 2 days after FUS-BBBO, in accordance with embodiments described herein;

FIG. 5e illustrates a graph including a sum of bioluminescence average radiance (p/s/cm²/sr) in five sections of each mouse 7 days after FUS-BBBO, in accordance with embodiments described herein;

FIG. 6a illustrates ex vivo bioluminescent imaging (BLI) of brain sections for measuring unmodified or engineered RLuc8.6 retention after delivery and ex vivo bioluminescent imaging representative data for mice at 2 days after FUS-BBBO; in accordance with embodiments of the present disclosure;

FIG. 6b illustrates ex vivo bioluminescent imaging (BLI) of brain sections for measuring unmodified or engineered RLuc8.6 retention after delivery and ex vivo bioluminescent imaging representative data for mice at 7 days after FUS-BBBO; in accordance with embodiments of the present disclosure;

FIG. 7a illustrates an embodiment directed to localized activation of dopaminergic cells with engineered AADC including an experimental scheme for assessing control of cellular signaling with RAID, in accordance with embodiments described herein;

FIG. 7b illustrates a graph directed to quantification of the AADC+ pixels in the FUS-targeted region among different groups, in accordance with embodiments described herein;

FIG. 7c illustrates representative immunostaining images of a coronal section from a mouse treated with FUS+AADC-IKVAV (SEQ ID NO: 23)+L-DOPA for AADC staining including one or more rectangular areas within the FUS target and contralateral site imaged at a higher magnification to compare AADC+ pixel counts, in accordance with embodiments described herein;

FIG. 7d illustrates representative immunostaining images of a coronal section adjacent to the area with the highest AADC-IKVAV (SEQ ID NO: 23) retention in a mouse treated with FUS+AADC-IKVAV (SEQ ID NO: 23)+L-DOPA, showing c-Fos, dopamine receptor D1R, and D2R staining including one or more rectangular areas within the FUS target and the contralateral site magnified for comparison of c-Fos+ cells with or without the expression of dopamine receptors D1+ or D2+, in accordance with embodiments described herein;

FIG. 7e illustrates a graph directed to quantification of c-Fos+ cells expressing dopamine receptors D1+ or D2+ in the FUS-targeted region, compared with contralateral site, in accordance with embodiments described herein;

FIG. 8a illustrates representative immunostaining images of AADC staining and spatial targeting precision for a plurality of mice in the study related to data presented in FIG. 4a-4c including images from mice treated with FUS+L-DOPA, in accordance with embodiments disclosed herein;

FIG. 8b illustrates representative immunostaining images of AADC staining and spatial targeting precision for a plurality of mice in the study related to data presented in FIG. 4a-4c including images from mice treated with FUS+AADC-IKVAV (SEQ ID NO: 23), in accordance with embodiments disclosed herein;

FIG. 8c illustrates representative immunostaining images of AADC staining and spatial targeting precision for a plurality of mice in the study related to data presented in FIG. 4a-4c including images from mice treated with FUS+AADC-IKVAV (SEQ ID NO: 23)+L-DOPA, in accordance with embodiments disclosed herein;

FIG. 8d illustrates a graph of spatial targeting precision of AADC-IKVAV (SEQ ID NO: 23) delivery based on its histological retention, in accordance with embodiments disclosed herein;

FIG. 8e illustrates a graph directed to a quantitative summary of AADC-IKVAV (SEQ ID NO: 23) retention in the CPu within the FUS-targeted region, in accordance with embodiments disclosed herein;

FIG. 9a illustrates AADC staining and representative immunostaining images of a coronal section from a mouse treated with FUS+L-DOPA, in accordance with embodiments of the present disclosure;

FIG. 9b illustrate c-Fos and dopamine receptor staining including representative immunostaining images of a coronal section adjacent to the area with the highest AADC-IKVAV (SEQ ID NO: 23) retention in a mouse treated with FUS+L-DOPA, in accordance with embodiments disclosed herein;

FIG. 9c illustrates representative immunostaining images of FIG. 9c at higher magnifications, in accordance with embodiments disclosed herein;

FIG. 9d illustrates AADC staining and representative immunostaining images of a coronal section from a mouse treated with FUS+AADC-IKVAV (SEQ ID NO: 23) for AADC staining, in accordance with embodiments disclosed herein;

FIG. 9e illustrates c-Fos and dopamine receptor staining including representative immunostaining images of a coronal section adjacent to the area with the highest AADC-IKVAV (SEQ ID NO: 23) retention in a mouse treated FUS+AADC-IKVAV (SEQ ID NO: 23), in accordance with embodiments disclosed herein;

FIG. 9f illustrates representative immunostaining images of FIG. 9e at higher magnifications, in accordance with embodiments disclosed herein;

FIG. 10 includes a graph (n=5 mice, P=0.9989, based on a Two-way ANOVA with Sidak's multiple comparison test) representing potential secondary effects of dopaminergic network activation, in accordance with embodiments of the present disclosure;

FIG. 11a illustrates embodiments of RAID control of behavior through targeted neuromodulation including an experimental scheme for RAID-induced control of motor behavior by targeted activation of dopamine neurons, in accordance with embodiments described herein;

FIG. 11b illustrates a graph of one or more mobile episodes measured in the open field test before and after administration of L-DOPA, in accordance with embodiments of the present disclosure;

FIG. 11c illustrates a graph of one or more counterclockwise rotations measured in the open field test before and after administration of L-DOPA, in accordance with embodiments of the present disclosure;

FIG. 11d illustrates a graph of one or more clockwise rotations measured in the open field test before and after administration of L-DOPA, in accordance with embodiments of the present disclosure;

FIG. 11e illustrates a graph directed to quantification of the AADC+ pixels in the FUS-targeted region (located at the left CPu) among one or more different groups, in accordance with embodiments of the present disclosure;

FIG. 11f illustrates representative immunostaining images of a coronal section adjacent to an area with the highest AADC-IKVAV (SEQ ID NO: 23) retention in a mouse treated with FUS+AADC-IKVAV (SEQ ID NO: 23)+L-DOPA including rectangular areas within the FUS target at the CPu and the contralateral site magnified to compare c-Fos+ cell counts, in accordance with embodiments of the present disclosure;

FIG. 11g illustrates a graph including data directed to quantification of c-Fos+ cells in the FUS targeted region at the CPu, compared with contralateral site, in accordance with embodiments of the present disclosure;

FIG. 12a illustrates a graph of mobile episodes before the administration of L-DOPA, in accordance with embodiments of the present disclosure;

FIG. 12b illustrates counterclockwise rotations before the administration of L-DOPA, in accordance with embodiments of the present disclosure;

FIG. 12c illustrates clockwise rotations before the administration of L-DOPA, in accordance with embodiments of the present disclosure;

FIG. 13 illustrates representative immunostaining images of AADC staining for all mice with AADC-IKVAV (SEQ ID NO: 23) delivery related to FIG. 5, in accordance with embodiments disclosed herein;

FIG. 14 illustrates magnification of representative immunostaining images of a coronal section from a mouse treated with FUS+AADC-IKVAV (SEQ ID NO: 23)+L-DOPA for AADC staining, in accordance with embodiments disclosed herein;

FIG. 15a illustrates weekly RAID-induced behavioral modulation including a first counterclockwise rotations graph measured in the open field test before and after administration of L-DOPA.

FIG. 15b illustrates weekly RAID-induced behavioral modulation including a second counterclockwise rotations graph measured in the open field test before and after administration of L-DOPA.

FIG. 15c illustrates weekly RAID-induced behavioral modulation including a first clockwise rotations graph measured in the open field test before and after administration of L-DOPA.

FIG. 15d illustrates weekly RAID-induced behavioral modulation including a second clockwise rotations graph measured in the open field test before and after administration of L-DOPA.

FIG. 16a illustrates embodiments directed to safety assessment of RAID protocol in behavior analysis including a histological classification of brain tissue damage at 25 FUS targeted sites in 25 mice following FUS-BBBO with or without AADC-IKVAV (SEQ ID NO: 23), in accordance with embodiments of the present disclosure;

FIG. 16b illustrates images of representative hematoxylin-stained brain section containing a small area of hemorrhage (indicated by a white arrow) in a mouse treated with FUS alone, in accordance with embodiments of the present disclosure;

FIG. 16c illustrates a graph directed towards quantification of GFAP+ cells in the FUS targeted region at striatum, compared with contralateral site, in accordance with embodiments of the present disclosure;

FIG. 16d is illustrative of a mouse body weight analysis including a weight just before FUS-BBBO or intravenous injection of unmodified or engineered RLuc8.6 and AADC-IKVAV (SEQ ID NO: 23) used to normalize the weight of each mouse during the experiment, in accordance with embodiments of the present disclosure;

FIG. 17 illustrates representative immunostaining images for GFAP in a mouse treated with FUS+AADC-IKVAV (SEQ ID NO: 23)+L-DOPA, in accordance with embodiments disclosed herein;

FIG. 18 is a graph directed to mouse body weight analysis after systemic administration of unmodified or engineered RLuc8.6, in accordance with embodiments described herein;

FIG. 19a is a schematic embodiment illustrating gene delivery-driven RAID modulation of behavior including an experimental scheme for AAV-driven RAID modulation of motor behavior through targeted delivery of AADC to striatal neurons, in accordance with embodiments of the present disclosure;

FIG. 19b illustrates one or more clockwise rotations in mice treated with an i.v. injection of AAV control measured in the open field test before and after L-DOPA administration, in accordance with embodiments of the present disclosure;

FIG. 19c illustrates one or more clockwise rotations in mice treated with an i.v. injection of FUS+AAV control measured in the open field test before and after L-DOPA administration, in accordance with embodiments of the present disclosure; and

FIG. 20 illustrates gene delivery-driven expression of the displayed RAID enzyme on the extracellular surface, in accordance with embodiments of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and enterprise-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

One or more embodiments are described herein directed to systems and methods to non-invasively test invasive therapies. Accordingly, a system is disclosed herein directed to Regionally Activated Interstitial Drugs (RAID). RAID provides noninvasive, multi-day, pharmacological control over specific sites of an intact brain. RAID may be implemented using focused ultrasound to noninvasively deliver engineered protein enzymes to one or more specific brain regions. The one or more specific brain regions include enzymes bound to the brain parenchyma for multiple days that may convert inert BBB-permeable prodrugs into active drugs. As such, described herein RAID is used to demonstrate systems and method to control neuronal function and behavior for several days, mimicking the effects of invasive drug delivery RAID is versatile and can be applied to various enzyme and prodrug pair to control various aspects of central nervous system physiology.

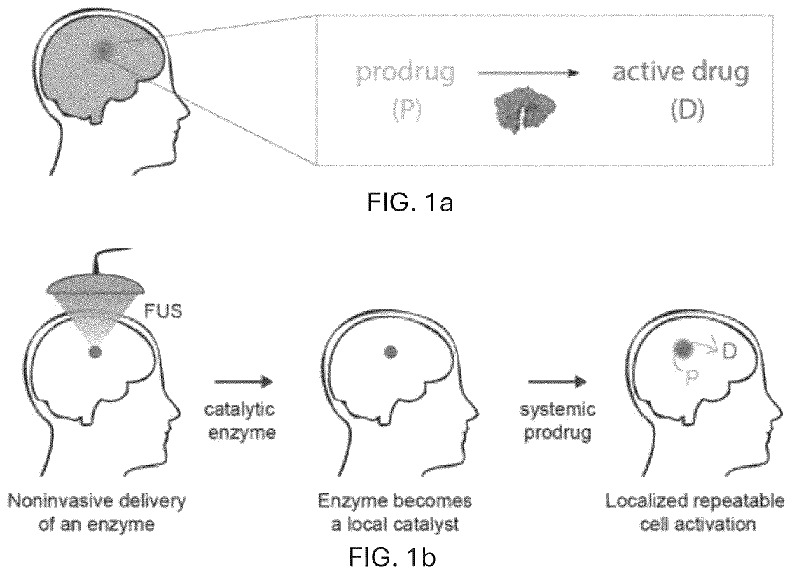

In some embodiments, RAID can be used to address current limitations related to site-specific brain therapeutics by offering noninvasive, site-, cell-type, and temporally specific neuromodulation that does not use genetically-encoded components. RAID enables multi-day, tunable neuromodulation using molecules compatible with FUS-BBBO delivery. In certain embodiments, RAID uses FUS-BBBO to deliver an engineered enzyme to a localized brain region. The engineered enzyme binds specifically to an interstitial space within the brain, where it may be retained for extended periods of time. When present, the engineered enzyme acts as a local drug factory (e.g., the engineered enzyme converts an inert systemically supplied and BBB-permeable prodrug into an active neuromodulatory drug) as discussed in reference to FIG. 1b below. In some embodiments, RAID may be tunable on demand. That is, by varying a dose of the prodrug, RAID can achieve stronger or weaker neuromodulation. Further, discussed herein one or more RAID enzymes stay within the brain for at least several days, and are capable of exerting spatially-specific neuromodulation and control of specific behaviors. Additionally and/or alternatively, gene delivery encoding the RAID enzyme offers prolonged expression and precise spatial control, enabling long-term and adaptable neuromodulation.

Previously available technology is limited as to noninvasively modulating a specific brain region. Additionally, while acoustically target chemogenetics may be used delivery of drugs (e.g., GABA) to specific brain regions is limited due to drugs being washed out within tens of minutes. As such, there is a need for improved techniques and methods. Embodiments disclosed herein long-term neuromodulation similar to ATAC, but without use of gene delivery are presented. In some embodiments, a type of protein-based biologic may be delivered to the brain using focused ultrasound. Disclosed systems and methods enable the protein-based biologic to remain in the brain interstitium for days. As such, systems and methods described herein may enable local production of neuroactive therapeutics. In certain embodiments, using these systems and methods may enable modulation of motor behavior and/or neuronal activity in one or more targeted regions for a series of days. A technical advantage of the disclosed embodiments includes delivery of molecules no larger than a protein, enabling use of safe focused ultrasound parameters, in contrast to previously available inefficient techniques used achieve long-term drug delivery with nanoparticles.

In some embodiments, systems and methods disclosed herein may be used as a therapy planning strategy. For example, it may be advantageous for a patient with epilepsy may be considering a resection to undergo an outpatient procedure and infusion of a biologic that could silence the targeted brain site on demand for several days. The silence of the target brain site may enable confirmation of a precision of seizure focus selection and would inform the patient of one or more potential side effects. Additionally and/or alternatively, embodiments disclosed herein may be used for neuroscience studies, particularly in large animals, wherein different parts of the brain may be noninvasively silenced or activated on various different days. However, controlling different brain regions for one or more days at a time is not feasible with current techniques of gene delivery due to the cost and formation of neutralizing antibody response after the first injection. Further, currently, there are no other technologies with a long-term action following focused ultrasound delivery using safe ultrasound pressures and using no gene therapy.

For example, previously available approaches include delivery of small molecule drugs with focused ultrasound induced blood-brain-barrier opening (FUS-BBBO). This approach may provide site-specific delivery of drugs, but the drugs do not stay put within the brain. For example, GABA only lasts around approximately 100 minutes. Approximately 100 minutes is not enough time for many real-world applications, where a patient would have to come back to the MRI suite to receive MRI opening every day. Alternative techniques may include FUS-BBBO delivery of nanoparticles that may provide sustained release of drugs over long periods of time. Unfortunately, nanoparticles are typically too large to enter through the pores in FUS-opened BBB which leads to poor delivery, use unsafe ultrasound pressures, or both. Contrary to previously available techniques, embodiments disclosed here achieve advantages of the small molecule delivery (e.g., safe pressures) while also providing long-term localized drug delivery. In this manner, RAID provides an improved system for site-specific brain therapeutics.

Depending on combinations of drugs and enzymes a particular drug or a particular enzyme may have systemic effects. Such systemic effects may be alleviated by targeting one or more particular receptors that are only present in the brain with prodrugs, or providing BBB impermeable inhibitor (e.g., carbidopa in case of AADC and L-Dopa delivery as disclosed herein). Presently disclosed systems and methods may enable localized, days-long control of specific brain regions. In some embodiments, system and methods described herein can be used for example to silence a site of potential resection for epilepsy surgery to identify if a suspected seizure focus reduces seizures and has tolerable side effects. In certain embodiments, RAID can be used be used in large and small animal studies. It should be noted, products and/or services may be developed using technology described herein including, but not limited to, different enzymes optimized to be retained within the brain, in combination with small molecule therapeutics that are modified by these enzymes to exert local therapeutic action.

With the preceding in mind, the following discussion provides further details and examples. Many brain therapies are invasive, expensive, and often irreversible, but their outcome is uncertain. For example, refractory epilepsy is often treated with surgical resection. However, up to 63% of patients continue experiencing seizures and a portion of patients may experience adverse neurological effects after surgery. One of the reasons for said adverse neurological effects may include difficulty in an accurate delineation of the seizure onset zone before surgical resection. Because seizures occur randomly and infrequently, patients are typically observed in an epilepsy unit for several days at a time, indicating a need for site-specific silencing of the seizure foci over multiple days. If such presumed seizure focus could be shut down before the surgery for sufficient length of time, it would enable identifying patients who would benefit from the surgery and improve the adherence to such therapy.

Additionally and/or alternatively, delivery of a gene encoding aromatic L-amino acid decarboxylase (AADC) was recently approved for treatment of genetic AADC deficiency. An ability to evaluate the optimal levels of dopamine production in each patient before gene therapy may be beneficial. Unfortunately, usually once the gene therapy is administered, it cannot be modified or readministered due to vector-directed immune response. Further, ability to control specific brain regions without deep-brain stimulation devices or invasive gene therapy may provide a non-surgical alternative to the patients suffering from various psychiatric and neurological disorders, but such therapy would have to be administered infrequently enough to allow these patients to lead normal lives.

Unfortunately, appropriate noninvasive methods for site-specific therapy is limited. Current techniques within the field of brain therapy may use small molecule drugs targeted to specific cellular receptors which diffuse throughout the brain contributing to off-target effects. Controlling specific brain sites with molecular precision may be done in research animals and has already catalyzed critical advances in neuroscience. However, the majority of these spatially specific neuromodulation tools rely on surgical delivery of molecules, implantation of devices, or gene delivery. While surgical delivery carries risks to patients and damages the tissues, gene therapy also runs the risk of vector-directed immune response and toxicity, and carries a high cost. For example, optogenetics can control neurons with tunable levels of activation, for extended periods of time, with spatial, cell-type, and temporal precision. However, optogenetics also requires gene delivery, usually an externally-powered device, and an implantation of optical fibers in the proximity of the stimulated site due to the limited penetration of light through the tissue. When optogenetics is used in large brain regions, it requires implantation of large numbers of such fibers leading to broad tissue damage, reducing its translational utility. Noninvasive long-term neuromodulation with spatial, cell-type, and temporal precision, and without an external wearable device is possible with Acoustically Targeted Chemogenetics (ATAC). However, ATAC may also include gene delivery to the brain with all its potential limitations and challenges. In ATAC, adeno-associated viral vectors (AAVs) are delivered systemically and then into specific brain regions with use of focused ultrasound blood-brain barrier opening (FUS-BBBO). These vectors encode chemogenetic receptors which may control the transduced neurons in response to a systemically administered drug. These approaches may have significant impact, however, in many cases there is a need to confirm the utility of targeting the specific brain site, and controlled molecular pathway before invasive devices, or expensive “single-use” gene therapy is administered.

Embodiments herein are directed to providing an improved method of providing small molecule delivery at safe pressures while also providing long-term localized drug delivery. A non-genetic approach that may be safely delivered to the brain with FUS-BBBO while also allowing for long-term drug action, molecular precision, and tunability of action is described herein. RAID is disclosed herein to provide a versatile noninvasive, site-, molecular-, and temporally-specific neuromodulation that does not involve challenges of genetically-encoded components or nanoparticles. FIG. 1a illustrates an embodiment of RAID directed to site-specific non-genetic therapeutics including an enzyme delivered to a selected site of brain specifically converts an inactive but BBB permeable prodrug (e.g., P) into a neuroactive drug (e.g., D), in accordance with embodiments described herein. RAID enables multi-day, tunable neuromodulation using molecules compatible with FUS-BBBO delivery as further described in reference to FIG. 1b. FIG. 1b illustrates an embodiment of RAID in which one or more catalytic enzymes are delivered to the brain via FUS-BBBO, wherein the catalytic enzymes bind within the brain interstitium to increase a residence time of the catalytic enzymes), in accordance with embodiments described herein. While the enzyme is present, neuronal activity around the enzyme can be modulated by conversion of a systemically supplied BBB-permeable prodrugs (P) into active drugs (D).

In certain embodiments, RAID, uses a FUS-BBBO to deliver an engineered enzyme to a localized brain region. The engineered enzyme binds to the interstitial space within the brain, where the engineered enzyme is retained for extended periods of time. When present, the engineered enzyme can act as a local drug factory. That is the engineered enzyme may convert an inert systemically supplied and BBB-permeable prodrug into an active drug as shown by FIG. 1b. RAID is compatible with various drug doses and is tunable on demand (e.g., by varying the dose of the prodrug). In some embodiments, RAID can be used to achieve stronger or weaker neuromodulation. As disclosed herein, RAID enzymes stay within the brain and remain functional for at least several days, depending on the enzyme. Additionally and/or alternatively, RAID may exert spatially-specific neuromodulation sufficient to control specific behaviors. RAID can be used to evaluate effects of site-specific modulation of neuronal activity within the brain. RAID is versatile and may be applied to different prodrug-enzyme pairs. RAID can be used to control one or more different signaling pathways, one or more cell types, or a combination thereof in various brain regions that may be targeted with FUS-BBBO. Further, FDA approved molecules and a protein naturally present in humans is used in the disclosed embodiments to showcase a potential utility of RAID in clinical research, therapy, therapy planning, or a combination thereof.

Retention of a Native Bioluminescent Protein in the Brain after FUS-BBBO Delivery

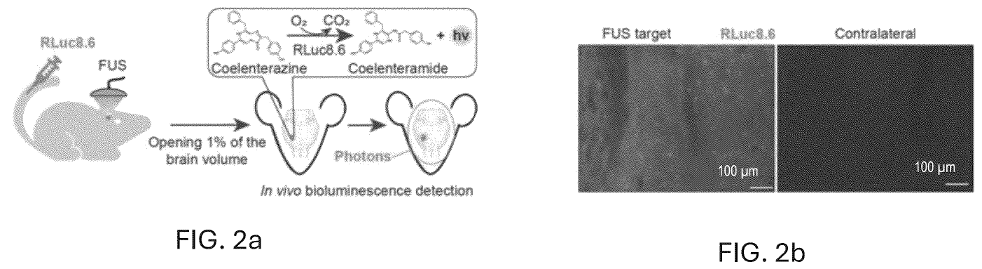

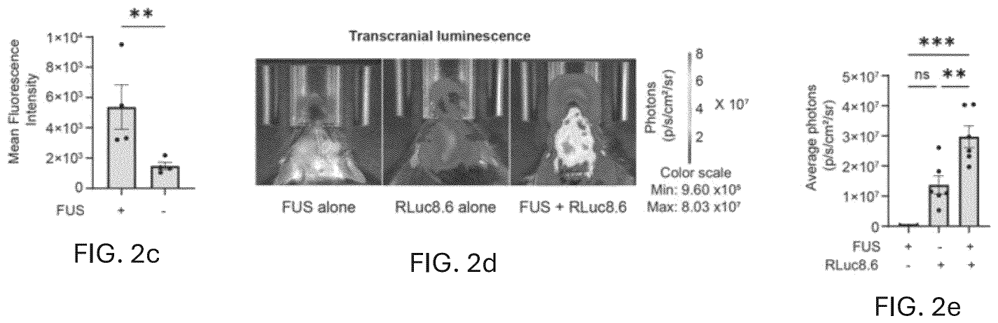

In some embodiments, evaluation of retention of enzymatic activity in the brain may be achieved through delivery of a model protein enzyme. The model protein enzyme is be detectable in vivo and allows for facile tracking of the kinetics of the model protein retention. In some embodiments, the model protein enzyme may include RLuc8.6 luciferase. RLuc8.6 luciferase converts coelenterazine substrate into coelenteramide. During conversion of coelenterazine substrate into coelenteramide one or more photons may be emitted. The photons can be noninvasively detected in the brain via an intravital imaging system (IVIS) equipment. Turning now to FIG. 2a, illustrates a schematic embodiment of a noninvasively delivered enzyme in a brain. As shown, the noninvasively delivered enzyme can retain activity in a mouse brain interstitium. FIG. 2a illustrates delivery of a recombinant luciferase (e.g., RLuc8.6) to the mouse brain through FUS-BBBO. The IVIS may be used to conduct bioluminescent imaging to assess an efficacy of targeted enzyme delivery and an activity of the targeted enzyme delivery. The activity is induced following an intraperitoneal injection of the Rluc8.6 substrate (e.g., coelenterazine (CTZ)). In this manner, in vivo bioluminescent detection of the coelenteramide in the mouse brain may be detected via the emitted photons.

Injection of a sample of purified RLuc8.6 (150 mg/kg) may be applied intravenously. FUS-BBBO may then be used to deliver the RLuc8.6 unilaterally into the caudate putamen (CPu) of mice. FIG. 2b and FIG. S1 illustrate confirmation of delivery of a spatially-specific by immunostaining in brain sections. FIG. 2b illustrates representative immunostaining images for RLuc8.6 in a mouse 1 h after FUS-BBBO. The immunostaining images targeting 4 sites in left striatum and systemic administration of RLuc8.6 (150 mg/kg) include one or more magnified views. A left image shows a FUS-targeted region (scale bar 100 μm). A right image shows a contralateral site (scale bar 100 μm).

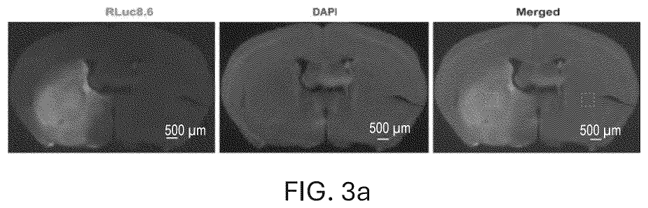

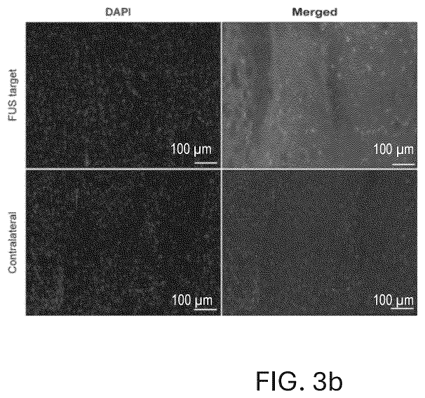

FIG. 3 illustrates additional representative immunostaining images after RLuc8.6 delivery related to FIG. 2b. FIG. 3a shows fluorescence imaging of coronal section with RLuc8.6 retention in a mouse 1 h after FUS-BBBO (targeting 4 sites in the left striatum) and systemic administration of RLuc8.6 (150 mg/kg), as shown in FIG. 2b. Cell nuclei were stained with DAPI. One or more areas of the FUS target and contralateral site in respective sections are shown by the one or more square areas in FIG. 3a. The scale bars correspond to 500 μm. The one or more square areas are imaged at a greater magnification to compare the average fluorescence intensity, as presented graphically in FIG. 2c. FIG. 3b illustrates the greater magnification to compare the average fluorescence intensity, illustrating a magnified view of FUS targeted region (upper images) and contralateral site (lower images) corresponding to the square areas in FIG. 3a. The scale bars correspond to 100 μm.

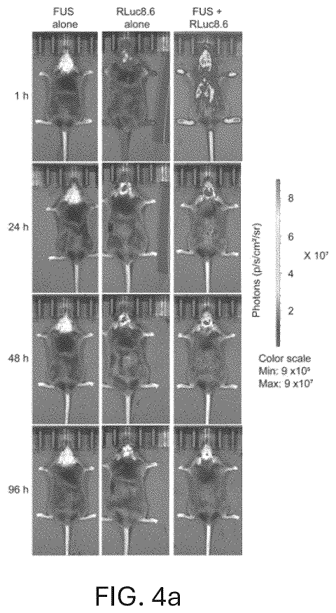

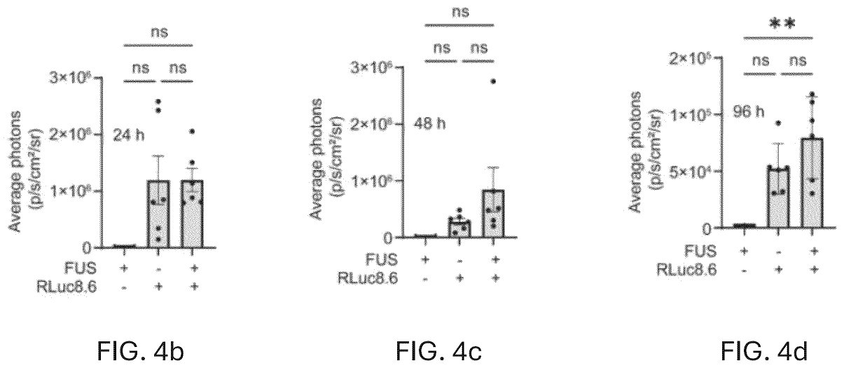

FIG. 2c illustrates an embodiment directed to quantification of average fluorescence intensity of RLuc8.6 over the FUS-targeted region and the contralateral site, in accordance with embodiments described herein. Data of FIG. 2c is presented as mean±s.e.m. n=4 mice for each group; **P<0.01, using ratio paired t test (two-tailed). As shown in FIG. 2c, a significantly higher immunostaining signal in the FUS target region (left striatum) at 1 h post-delivery was found compared to the untargeted contralateral site. To evaluate whether RLuc8.6 retained enzymatic activity in the brain interstitium, in vivo activity of RLuc8.6 at 4 different timepoints was measured using IVIS. Detectable RLuc8.6 enzymatic activity enhanced by FUS-BBBO in the brain after 1 h was found as shown by FIG. 2d. FIG. 2d illustrates an embodiment directed to in vivo bioluminescent imaging representative data at field of view A (3.9 cm across), wherein FUS targeting one site at left striatum was followed by an IV injection of RLuc8.6 (8 mg/kg) or PBS buffer control. RLuc8.6 alone group was intravenously injected with the same dose (8 mg/kg) of RLuc8.6 without FUS-BBBO procedure. After 1-hour, bioluminescent imaging was performed after administration of CTZ (i.p., 3.5 mg/kg). Further, FIG. 2e demonstrates that opening the BBB in 1% of the brain volume led to 2.2(±0.3)-fold higher signal when averaged over the entire brain compared to RLuc8.6 alone without FUS. As shown, FIG. 2e illustrates a quantification of bioluminescence average radiance (p/s/cm²/sr) at field of view C (13 cm across) in the head region 1 h after FUS-BBBO. Data is presented as mean±s.e.m. n=6 mice for RLuc8.6 alone and FUS+RLuc8.6 groups, n=3 for FUS alone group; **P<0.01, ***P<0.001, ns (not significant), using one-way ANOVA with Tukey's honestly significant difference test. Considering the calculated volume of the single BBB opening site compared to the whole brain volume, the upper threshold of delivery should equal approximately 220-fold enzyme concentration increase within the targeted site compared to untargeted controls, which is consistent with previous studies of FUS-BBBO delivery for molecules of this size. However, the IVIS signal decayed over time, and the enzyme levels were statistically indistinguishable from controls from 24 hours after FUS-BBBO onwards as illustrated by FIG. 4. FIG. 4 includes representative images and data of in vivo BLI at different time points after delivering RLuc8.6. FIG. 4a illustrates in vivo bioluminescent imaging representative data at field of view C (13 cm) for the mice shown in FIG. 2d, 1 h, 24 h, 48 h and 96 h after IV injection of RLuc8.6 (8 mg/kg) or PBS buffer with or without insonation. The mice were administered CTZ (i.p., 3.5 mg/kg) before each bioluminescent imaging. FIG. 4b-4d include graphs directed to quantification of bioluminescence average radiance (p/s/cm²/sr) at field of view C (13 cm) in the head region 24 h, 48 h and 96 h after FUS-BBBO. Data is presented as mean±s.e.m. n=6 mice for RLuc8.6 alone and FUS+RLuc8.6 groups, n=3 for FUS alone group; **P=0.0045, ns (not significant), using One-way ANOVA test. It should be noted, data presented in FIG. 2a-e, FIG. 3, and FIG. 4 revealed that the enzyme retains activity in the brain following RAID protocol, but the activity decays over the next 24 hours.

### Protein Engineering Improves the Retention of Delivered Enzyme

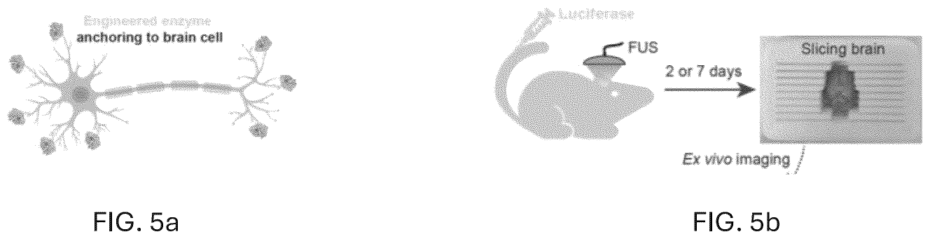

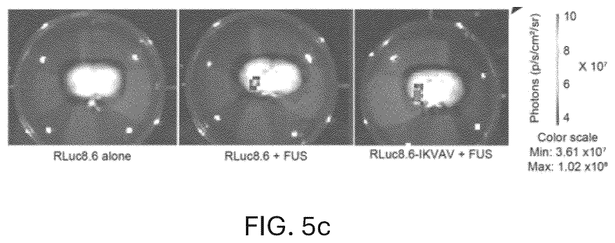

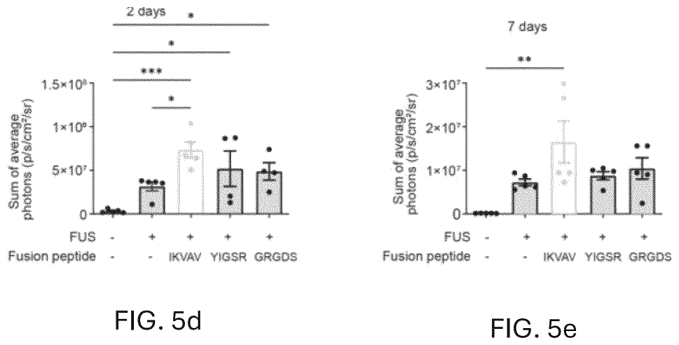

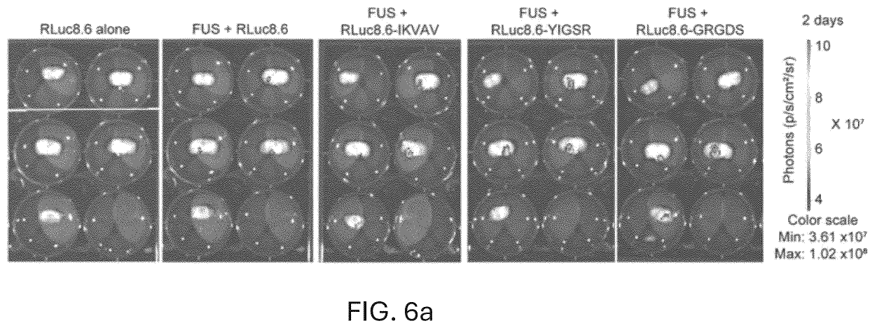

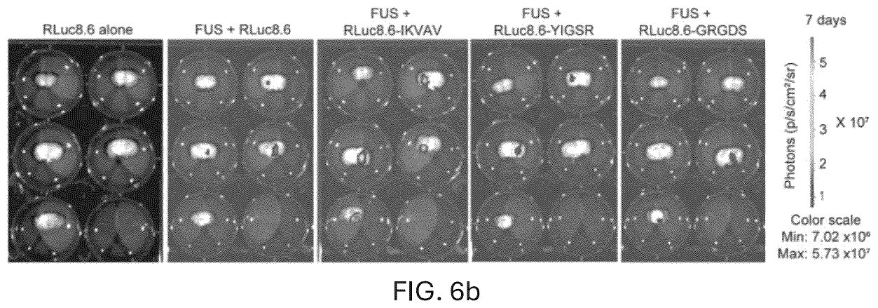

In some embodiments, enzyme's clearance out of the brain interstitium can lead to a reduction of signal over time. As such, engineering RAID enzymes that bind to the cells in the brain to improve the enzymes' retention may be advantageous. FIG. 5a illustrates an embodiment directed towards engineering an enzyme to improve retention in a brain including a schematic diagram of engineered cell-adhesive peptides that mimic the ECM for attaching prodrug enzymes to brain cells, in accordance with embodiments described herein. RLuc8.6 may be fused to engineered cell-adhesive peptides. The engineered cell-adhesive peptides mimic the extracellular matrix (ECM). The engineered cell-adhesive peptides have been shown to promote attachment to specific cells targeting cell-surface receptors. To improve the enzyme retention after delivery and brain-tissue specificity, the RLuc8.6 may be fused to IKVAV (SEQ ID NO: 23), GRGDS (SEQ ID NO: 24), and YIGSR (SEQ ID NO: 25) (e.g., known to bind to neurons). To avoid skull attenuating the bioluminescent signal, the retention of each construct post-mortem in 2 mm thick unfixed tissue sections can be analyzed. Two time points, 2 and 7 days after FUS-BBBO procedure were tested as shown in FIG. 5b. FIG. 5b illustrates an embodiment of an experimental scheme for measuring the retention of unmodified or engineered RLuc8.6 in a mouse brain. In some embodiments, FUS-BBBO targeting three sites of left brain in mice following IV injection of RLuc8.6 (100 mg/kg) or engineered variant (104 mg/kg) and microbubbles was conducted. RLuc8.6 alone group was only intravenously injected with the same dose of RLuc8.6 without insonation. After 2 or 7 days, mouse brains were extracted without perfusion and cut into 2 mm slices before bioluminescent imaging. In some embodiments, FUS-mediated delivery led to over 10-fold higher level of bioluminescence over no-FUS control group as illustrated by FIG. 5c-5e and FIG. 6. It should be noted, tested enzyme variants demonstrated significant improvement in retention at 2 days.

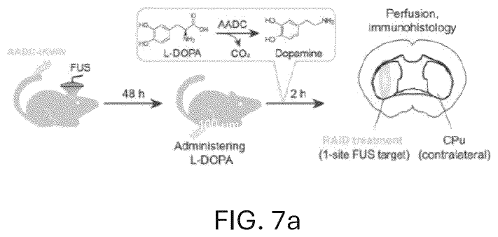

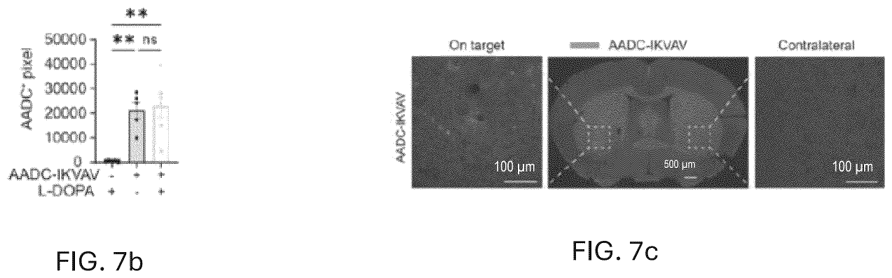

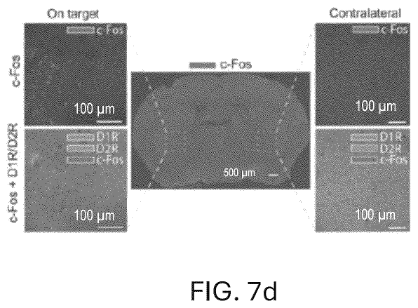

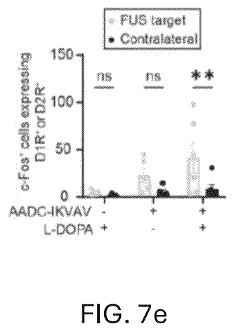

FIG. 5c illustrates ex vivo bioluminescent imaging representative data for mice at 2 days after FUS-BBBO, in accordance with embodiments described herein. After cutting, each 2 mm brain section are individually transferred into a 6-well glass bottom plate filled with 2 mL PBS buffer. BLI at field of view C (13 cm) was conducted immediately after adding 1 mL dissolved CTZ with a final concentration of 10 PM. FIG. 5d illustrates a graph including a sum of bioluminescence average radiance (p/s/cm²/sr) in five sections of each mouse 2 days after FUS-BBBO, in accordance with embodiments described herein. Data provided in FIG. 5d is n=4/5 mice per group, P=0.0006, 0.0251 and 0.0387 for IKVAV (SEQ ID NO: 23), YIGSR (SEQ ID NO: 25) and GRGDS (SEQ ID NO: 24) fused RLuc8.6 respectively, One-way ANOVA with Tukey's HSD test). FIG. 6 illustrates ex vivo bioluminescent imaging (BLI) of brain sections for measuring unmodified or engineered RLuc8.6 retention after delivery and ex vivo bioluminescent imaging representative data for mice at 2 days (FIG. 6a) and 7 days (FIG. 6b) after FUS-BBBO. BLI is performed at field of view C (13 cm across) immediately after adding 1 mL dissolved CTZ with a final concentration of 10 μM.

In certain embodiments, fusing RLuc8.6 with one of the ECM-mimicking peptides (IKVAV (SEQ ID NO: 23)) enhanced enzyme activity 2 days after FUS-BBBO delivery when compared to unmodified RLuc8.6 at 2-days as presented by FIG. 5d. FIG. 5d illustrates a graph including a sum of bioluminescence average radiance (p/s/cm²/sr) in five sections of each mouse 2 days after FUS-BBBO, in accordance with embodiments described herein. Fusion of Rluc8.6 and IKVAV (SEQ ID NO: 23) (RLuc8.6-IKVAV) showed highest mean activity improvement at 2.3(±0.3)-fold over unmodified RLuc8.6 (n=5 mice per group, P=0.0452, One-way ANOVA with Tukey's HSD test). The RLuc8.6-IKVAV (SEQ ID NO: 23) was also the only RAID enzyme that was significantly different than RLuc8.6 alone control at 7-days after FUS-BBBO as shown by FIG. 5e. FIG. 5e illustrates a graph including a sum of bioluminescence average radiance (p/s/cm²/sr) in five sections of each mouse 7 days after FUS-BBBO, in accordance with embodiments described herein. Data is presented as mean±s.e.m. n=5 mice in each group except for RLuc-8.6-YIGSR (SEQ ID NO: 25) and RLuc8.6-GRGDS (SEQ ID NO: 24) groups at 2 days (n=4), since 2 mice from RLuc-8.6-YIGSR (SEQ ID NO: 25) and RLuc-8.6-GRGDS (SEQ ID NO: 24) groups exhibited FUS-BBBO-related tissue damage with abnormally high bioluminescence levels, leading to their exclusion from the analysis. *P<0.05, **P<0.01, ***P<0.001, using One-way ANOVA with Tukey's honestly significant difference test. It should be noted, IKVAV (SEQ ID NO: 23) fusion enhanced the RLuc8.6 retention, resulting in 113(±33)-foldhigher level of bioluminescence than the control group without applying FUS (n=5 mice per group, P=0.0012, One-way ANOVA with Tukey's HSD test). Additionally, RLuc8.6 is shown to remain functional within the brain parenchyma even at the 7-day timepoint.

### Spatiotemporal Modulation of Dopamine Receptor-Expressing Brain Cells

In some embodiments, a feasibility of using RAID for localized neuromodulation is determined. RAID can be theoretically applied to any enzyme-prodrug pair as long as the enzyme-prodrug pair uses BBB-permeable prodrug. Thus, RAID is a flexible paradigm that does not depend on targeting specific molecular pathways. In certain embodiments, a well-validated system with a clinically used prodrug and an enzyme that occurs naturally within the brain, and where any peripheral effects can be suppressed with a non-BBB permeable drug was chosen for analysis. Specifically, an L-DOPA may be selected as a prodrug. The L-DOPA is be converted to a neurotransmitter dopamine through a protein enzyme such as Aromatic-L-amino-acid decarboxylase (AADC). Dopamine is involved in multiple aspects of the brain function including motor control, reward, and motivation. Since L-DOPA exists naturally in the healthy brain, AADC is expected to have some background activity, which theoretically can allow for the enzyme delivery alone to have measurable effects. As such, exploration of the enzyme-prodrug pair may have a high translational significance based on relevance to investigation of natural dopaminergic circuitry. In this manner, such exploration may have potential for the treatment of Parkinson's Disease, where low amount of cellular AADC in basal ganglia limits dopamine production. Peripheral effects of L-DOPA may be suppressed by administration of non-BBB-permeable carbidopa to avoid peripheral effects. Without wishing to be bound by theory, while AADC is naturally present in some brain regions, locally increasing AADC concentration can sensitize the targeted brain region to lower doses of L-DOPA. Such sensitization may lead to a distinguishable site-specific neuromodulation that may be used in, for example, investigating the dopaminergic circuitry, testing the suitability of the brain region for AADC-expressing gene therapy, lowering the dose of L-DOPA necessary to treat PD, or a combination thereof. As such, sensitization can be used to distinguish associated side effects.

In some embodiments, disclosed techniques may provide a proof-of-concept for use of RAID, and a tool to evaluate effects of local perturbations of the dopaminergic network. It should be noted, the dopamine receptor-expressing brain cells in striatum was selected for activation. The dopamine receptor-expressing brain cells are therapeutically relevant brain region already targeted for gene therapy for AADC deficiency and Parkinson's disease. However, it should be noted one or regions may be targeted using the techniques described herein. In certain embodiments, to improve AADC's retention within the brain, AADC can be fused with IKVAV (SEQ ID NO: 23) as discussed above in reference to FIG. 5d-5e.

FIG. 7a illustrates an embodiment directed to localized activation of dopaminergic cells with engineered AADC including an experimental scheme for assessing control of cellular signaling with RAID, in accordance with embodiments described herein. AADC-IKVAV (SEQ ID NO: 23) (250 mg/kg) or PBS buffer are be injected IV and are followed with FUS-BBBO targeting left Caudate Putamen (CPu). After 2 days, L-DOPA (2 mg/kg, i.p.) or vehicle are administered, and 2 h later perfused the mice to analyze AADC-IKVAV (SEQ ID NO: 23) retention and neuronal activation as indicated by c-Fos expression in tissue sections. In some embodiments, a sample of purified AADC-IKVAV (SEQ ID NO: 23) (250 mg per kg of body weight) is injected intravenously. FUS-BBBO is targeted to a single site in the left caudate putamen (CPu) of wild-type mice. After 48 h, mice receive an intraperitoneal (IP) injection of L-DOPA (2 mg/kg) and two hours later are perfused to evaluate spatial precision and presence of AADC-IKVAV (SEQ ID NO: 23) delivery in tissue sections.

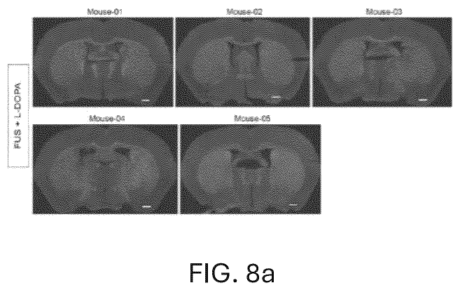

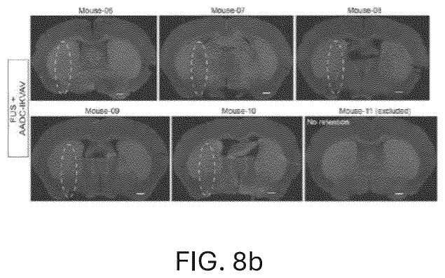

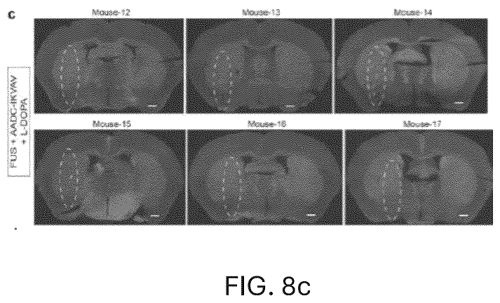

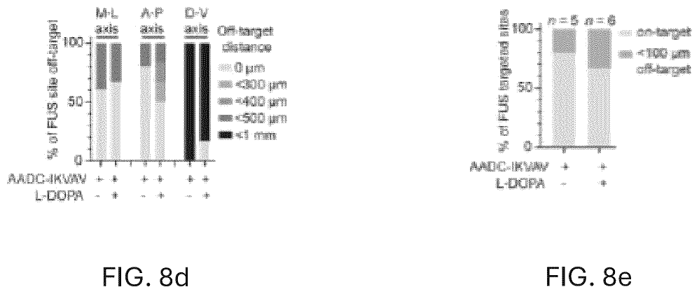

As shown in FIG. 8, AADC-IKVAV (SEQ ID NO: 23) can be retained at the FUS-targeted site following FUS-BBBO delivery, and administration of L-DOPA may have no effect on that retention. FIG. 8 illustrates representative immunostaining images of AADC staining and spatial targeting precision for a plurality of mice in the study related to data presented in FIG. 7a-7c. FIG. 8a includes representative immunostaining images of coronal sections from mice treated with FUS+L-DOPA. FIG. 8b includes representative immunostaining images of coronal sections from mice treated with FUS+AADC-IKVAV (SEQ ID NO: 23). FIG. 8c includes representative immunostaining images of coronal sections from mice treated with FUS+AADC-IKVAV (SEQ ID NO: 23)+L-DOPA. Images provided in FIG. 8a-8c illustrate sections with a highest AADC staining signals around the striatum area in each mouse. It should be noted, mouse-11 in the FUS+AADC-IKVAV (SEQ ID NO: 23) group was excluded due to lower AADC+ pixel counts compared to the FUS+L-DOPA group's average and no AADC-IKVAV (SEQ ID NO: 23) retention. The scale bar corresponds to 500 μm. A presumed actual FUS target region for the mice with AADC-IKVAV (SEQ ID NO: 23) retention was labeled using a theoretical ultrasound beam (represented by a white dashed line in FIG. 8). A size of the presumed actual FUS target region was estimated from the image scale bar and the dimensions of the ultrasound transducer, which had axial and lateral diameters of 5 mm and 1.2 mm, respectively. Mouse-15's AADC-IKVAV (SEQ ID NO: 23) retention is indicated by a white arrow for easy identification. FIG. 8d includes a graph of spatial targeting precision of AADC-IKVAV (SEQ ID NO: 23) delivery based on its histological retention. The estimation of distance from the intended target was made by comparison to the target programmed within the atlas embedded in the RK-50 ultrasound device. Displacement of less than 300 μm distance was considered accurate. n=5 mice for FUS+AADC-IKVAV (SEQ ID NO: 23) and n=6 mice for FUS+AADC-IKVAV (SEQ ID NO: 23)+L-DOPA group. M-L represents the medio-lateral axis, A-P represents the anterior-posterior axis, and D-V represents the dorso-ventral axis. FIG. 8e includes a graph directed to a quantitative summary of AADC-IKVAV (SEQ ID NO: 23) retention in the CPu within the FUS-targeted region. It should be noted, if there was no retention in the CPu, the distance of retention to the nearest CPu region was estimated using the same methodology as in point d.

In some embodiments, mice treated with AADC-IKVAV (SEQ ID NO: 23), with or without subsequent L-DOPA administration, exhibited locally elevated AADC levels that were 38.5 (±6.1)-fold and 41.8 (±8.9)-fold higher, respectively, compared to the FUS alone control group. FIG. 7b illustrates a graph directed to quantification of the AADC+ pixels in the FUS-targeted region among different groups, in accordance with embodiments described herein. Data is presented as mean±s.e.m. n=5 mice for FUS alone+L-DOPA and FUS+AADC-IKVAV (SEQ ID NO: 23) groups, n=6 mice for FUS+AADC-IKVAV (SEQ ID NO: 23)+L-DOPA; P=0.0056 and P=0.0021, **P<0.01, ns (not significant), using One-way ANOVA with Tukey's honestly significant difference test. One mouse in the FUS+AADC-IKVAV (SEQ ID NO: 23) group was excluded due to no observed AADC-IKVAV (SEQ ID NO: 23) retention. FIG. 7c illustrates representative immunostaining images of a coronal section from a mouse treated with FUS+AADC-IKVAV (SEQ ID NO: 23)+L-DOPA for AADC staining including one or more rectangular areas within the FUS target and contralateral site imaged at a higher magnification to compare AADC+ pixel counts, in accordance with embodiments described herein.

Further, the spatial targeting precision of AADC-IKVAV (SEQ ID NO: 23) delivery based on the site of AADC delivery as compared to the targeted site in a brain atlas embedded within the RK-50 ultrasound device is assessed. Among the 11 mice receiving AADC-IKVAV (SEQ ID NO: 23), 64% achieved the FUS target alignment in both medio-lateral and anterior-posterior axes as shown in FIG. 8d, with displacement of less than 300 microns as measured between the center of a simulated FUS beam profile in the histological section image and a brain atlas. For mice showing mistargeting, the displacement ranged from 300 to 1000 μm in any axis, resulting in detectable AADC-IKVAV (SEQ ID NO: 23) retention within the CPu for 73% of tested mice, while the remaining 27% showed efficient retention within 100 μm of the CPu as shown in FIG. 8e.

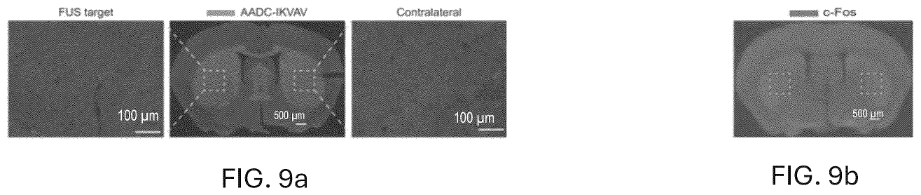

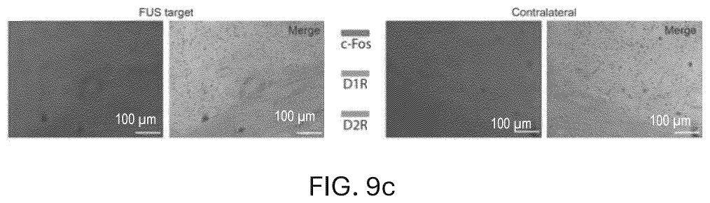

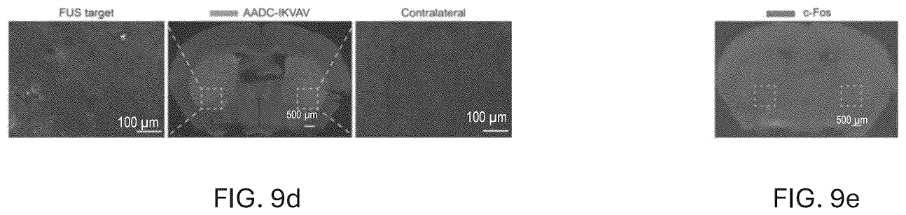

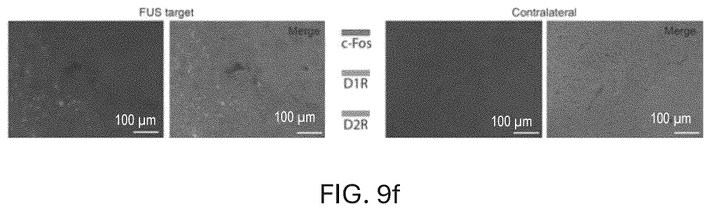

In some embodiments, to evaluate whether RAID with AADC and L-DOPA may elicit local cellular signaling the target cells that express dopamine receptors D1 and D2 that are found in the CPu can be analyzed. A significant increase in c-Fos in cells showing D1- or D2-positive immunostaining in mice that were subjected to the full RAID treatment is shown in FIG. 7d, 7e and FIG. 9. FIG. 7d illustrates representative immunostaining images of a coronal section adjacent to the area with the highest AADC-IKVAV (SEQ ID NO: 23) retention in a mouse treated with FUS+AADC-IKVAV (SEQ ID NO: 23)+L-DOPA, showing c-Fos, dopamine receptor D1, and D2 staining including one or more rectangular areas within the FUS target and the contralateral site magnified for comparison of c-Fos+ cells with or without the expression of dopamine receptors D1+ or D2+, in accordance with embodiments described herein. FIG. 7e illustrates a graph directed to quantification of c-Fos+ cells expressing dopamine receptors D1+ or D2+ in the FUS-targeted region, compared with contralateral site, in accordance with embodiments described herein. Data is presented as mean±s.e.m. n=5 mice for FUS alone+L-DOPA and FUS+AADC-IKVAV (SEQ ID NO: 23) group, n=6 mice for FUS+AADC-IKVAV (SEQ ID NO: 23)+L-DOPA; **P<0.01, ns (not significant), using Two-way ANOVA with Sidak's multiple comparison test.

FIG. 9 illustrates representative immunostaining images of control groups related to FIG. 7a and FIG. 7d. FIG. 9a includes AADC staining and representative immunostaining images of a coronal section from a mouse treated with FUS+L-DOPA. FIG. 9d includes AADC staining and representative immunostaining images of a coronal section from a mouse treated with FUS+AADC-IKVAV (SEQ ID NO: 23) for AADC staining. As shown, by one or more rectangular areas within the FUS target and contralateral site of FIG. 9a, were imaged at a higher magnification to compare AADC+ pixel counts. The scale bars correspond to 500 μm (for the entire coronal section) and 100 μm (for the magnified view), respectively. FIG. 9b and FIG. 9c include c-Fos and dopamine receptor staining including representative immunostaining images of a coronal section adjacent to the area with the highest AADC-IKVAV (SEQ ID NO: 23) retention in a mouse treated with FUS+L-DOPA. FIG. 9e and FIG. 9f include c-Fos and dopamine receptor staining including representative immunostaining images of a coronal section adjacent to the area with the highest AADC-IKVAV (SEQ ID NO: 23) retention in a mouse treated FUS+AADC-IKVAV (SEQ ID NO: 23), showing c-Fos, dopamine receptor D1R, and D2R staining. Rectangular areas of FIG. 9b and FIG. 9e within the FUS target and the contralateral site were magnified for the comparison of c-Fos+ cells with or without the expression of dopamine receptors D1+ or D2+. The scale bars correspond to 500 μm (FIG. 9b and FIG. 9e) and 100 μm (FIG. 9c and FIG. 9f), respectively.

In some embodiments, as shown in FIG. 7e a 6.6 (±1.7)-fold higher count in the FUS-targeted region compared to the contralateral site (n=6 mice, P=0.0052, as determined by a Two-way ANOVA with Sidak's multiple comparison test). No significant change was observed in the control groups, either those treated with FUS alone+L-DOPA (n=5 mice, P=0.9845, based on a Two-way ANOVA with Sidak's multiple comparison test) or those treated with FUS+AADC-IKVAV (SEQ ID NO: 23) without L-DOPA (n=5 mice, P=0.2046, as determined by a Two-way ANOVA with Sidak's multiple comparison test). As such, RAID can be used to specifically modulate activity of dopamine-responsive cells, while effects are specific to the FUS-targeted hemisphere.

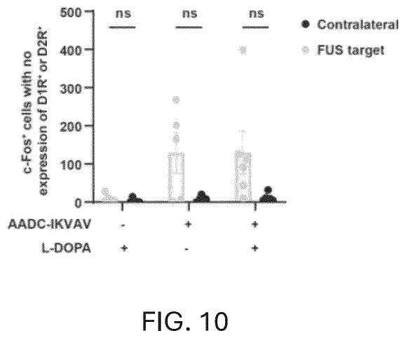

It should be noted, while FUS-BBBO on CPu was analyzed, some amount of AADC delivery may also present in off-target areas, as would be expected given the size of FUS-BBBO in our equipment (ovoid with ˜1×5 mm major axes diameters). Regions of the brain are highly interconnected with neighboring and midbrain neurons, allowing for potential secondary activation or inhibition of neuronal activity. However, no significant changes in the group of mice that received only FUS-BBBO without AADC-IKVAV (SEQ ID NO: 23) delivery were found as illustrated by FIG. 10. FIG. 10 includes a graph (n=5 mice, P=0.9989, based on a Two-way ANOVA with Sidak's multiple comparison test) representing potential secondary effects of dopaminergic network activation. Quantification of c-Fos+ cells with no expression of dopamine receptors D1+ or D2+ in the FUS-targeted region in the vicinity of CPu, compared with contralateral site. Data are presented as mean±s.e.m. n=5 mice for FUS alone+L-DOPA and FUS+AADC-IKVAV (SEQ ID NO: 23) groups, n=6 mice for FUS+AADC-IKVAV (SEQ ID NO: 23)+L-DOPA; ns (not significant), using Two-way ANOVA test. It should be noted, no statistically significant changes were found in these regions in mice receiving FUS+AADC-IKVAV (SEQ ID NO: 23) alone (n=5 mice, P=0.0832, based on a Two-way with Sidak's multiple comparison ANOVA test), or FUS+AADC-IKVAV (SEQ ID NO: 23)+L-DOPA (n=6 mice, P=0.0571, based on a Two-way ANOVA with Sidak's multiple comparison test).

### Behavioral Validation of RAID

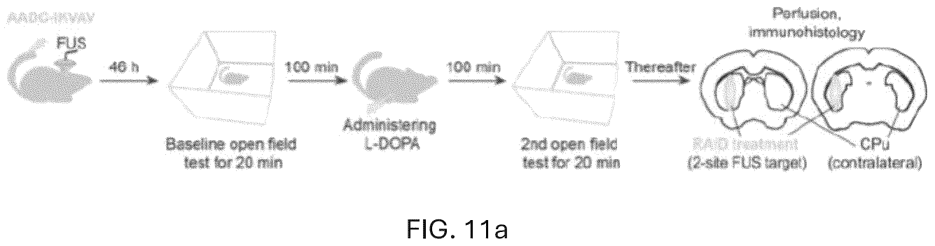

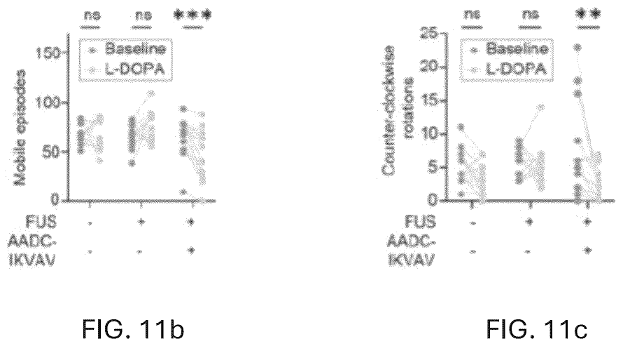

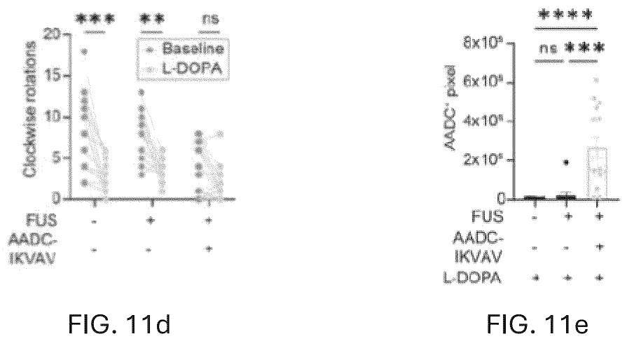

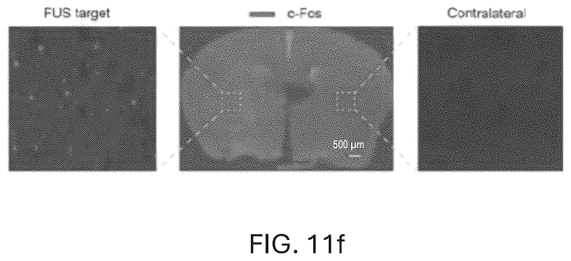

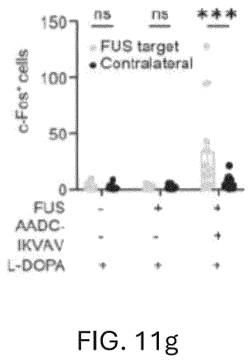

RAID neuromodulation provides an approach for precise modulation of brain activity through regionally activated interstitial drug delivery. It should be noted, site-specific, noninvasive methods hold promise for controlled behavioral modulation. As such, to evaluate long-term efficacy of RAID neuromodulation, RAID's ability to sustain behavioral control over time is described herein. In some embodiments, to measure effects of RAID neuromodulation on behavior, targeting dorsal striatum with two FUS sites are be analyzed. In some instances, effects of RAID neuromodulation by targeting dorsal striatum with two FUS sites can be used to increase an amount of on-target delivery of AADC in the CPu region that contains dopamine-responsive cells. Dopamine is involved in regulation of motor behavior, and previous studies indicate that localized, unilateral, delivery of dopamine to striatum resulted in changes to locomotor activity and rotational behavior. With this in mind, in certain embodiments, a pair of AADC-IKVAV (SEQ ID NO: 23) and L-DOPA may be used to evaluate whether RAID can induce similar behavioral changes after the prodrug administration. In some embodiments, AADC-IKVAV (SEQ ID NO: 23) is delivered via FUS-BBBO to two sites in the left striatum region of wild type mice. After 46 h mice were placed in an open field (OF) to test their locomotor activity for 20 minutes as shown in FIG. 11a. FIG. 11a illustrates embodiments of RAID control of behavior through targeted neuromodulation including an experimental scheme for RAID-induced control of motor behavior by targeted activation of dopamine neurons, in accordance with embodiments described herein. As illustrated, FUS in two sites of the left striatum are used to deliver AADC-IKVAV (SEQ ID NO: 23) (250 mg/kg, i.v. injection) or PBS buffer control. After 2 days, open field test (OFT) was performed twice before and after a single injection of the prodrug L-DOPA (2 mg/kg, i.p.), allowing 200 minutes between each test. The locomotor activity was recorded for 15 min after a habituation period of 5 min in each test. After the testing, mice were perfused 2 h after L-DOPA administration for histologic examination of AADC-IKVAV (SEQ ID NO: 23) retention and neuron activation (c-Fos+) to confirm localize neuromodulation. As schematically depicted in FIG. 11a, the AADC protein fused with a short peptide IKVAV (SEQ ID NO: 23) (e.g., AADC-IKVAV (SEQ ID NO: 23)) to two sites in the left striatum of wild-type mice using FUS-BBBO. After 46 hours, the mice underwent an open field (OF) test to assess baseline locomotor activity over a 20-minute period. The mice were then returned to their home cages and, 100 minutes later, received an intraperitoneal injection of L-DOPA (2 mg/kg) before undergoing another OF test.

FIG. 11b illustrates a graph of one or more mobile episodes measured in the open field test before and after administration of L-DOPA, in accordance with embodiments of the present disclosure. As shown, RAID treatment showed 38±8% decrease in mobile episodes, where mice move after an extended period of freezing (longer than 2 s) (n=14 mice, P=0.0005, Two-way ANOVA with Sidak's multiple comparison test). It should be noted, the decrease is in line with previous studies showing that direct unilateral injection of dopamine into the striatum led to a reduction in locomotor activity (e.g., moving, walking). However, no significant change in episodes of mobility was observed in the control groups treated with either FUS alone or wild type mice. FIG. 11c illustrates a graph of one or more counterclockwise rotations measured in the open field test before and after administration of L-DOPA, in accordance with embodiments of the present disclosure. As shown in FIG. 11 cRAID induce contralateral rotations, as previously demonstrated with direct unilateral dopamine injections into the caudate putamen. L-DOPA reduce counter-clockwise (ipsilateral) rotations by 63±10% in mice with full RAID treatment (n=14 mice, P=0.0018, Two-way ANOVA with Sidak's multiple comparison test) but not in the control mice with FUS alone or no treatment at all (n=11 mice for FUS alone group, n=12 mice for wild-type group; P=0.9992 and 0.6019 respectively, Two-way ANOVA with Sidak's multiple comparison test).

FIG. 11d illustrates a graph of one or more clockwise rotations measured in the open field test before and after administration of L-DOPA, in accordance with embodiments of the present disclosure. Data is presented as mean±s.e.m. n=12 mice for wild-type group, n=11 mice for FUS alone group and n=14 mice for FUS+AADC-IKVAV (SEQ ID NO: 23) group; **P<0.01, ***P<0.001, ns (not significant), using Two-way ANOVA with Sidak's multiple comparison test. In contrast to counterclockwise rotations, the clockwise (contralateral) rotation showed an opposite trend. The untreated and FUS alone groups showed decreased clockwise rotation after injection of L-DOPA by 52±8% and 44±13% (n=12 mice for wild-type group, n=11 mice for FUS alone group; P=0.0001 and P=0.0011 respectively, Two-way ANOVA with Sidak's multiple comparison test), but RAID treatment prevented that decrease (n=14 mice, P=0.2951, Two-way ANOVA with Sidak's multiple comparison test).

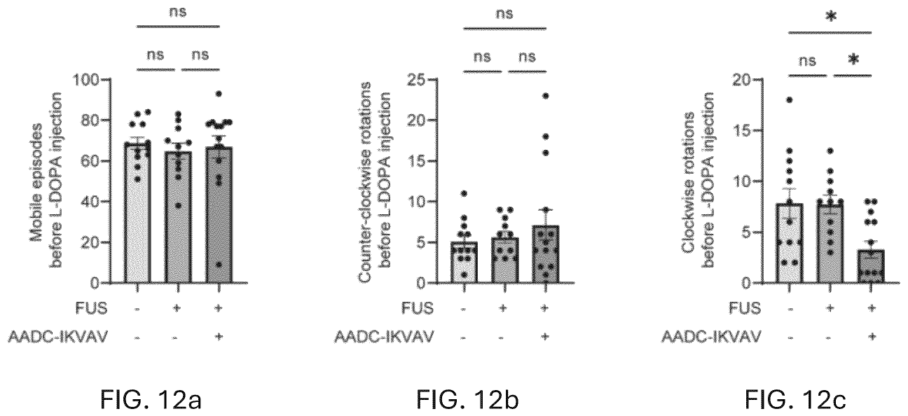

In some embodiments, to evaluate whether the AADC-IKVAV (SEQ ID NO: 23) alone had any effects on mouse behavior, the motor behavior of mice in the open field before L-DOPA administration may be compared. FIG. 12 illustrates a series of graphs to compare a series of groups prior to administration of L-DOPA. FIG. 12a illustrates mobile episodes before the administration of L-DOPA. FIG. 12b illustrates counterclockwise rotations before the administration of L-DOPA. FIG. 12c illustrates clockwise rotations before the administration of L-DOPA. Data is presented as mean±s.e.m. n=12 mice for wild-type group, n=11 mice for FUS alone group and n=14 mice for FUS+AADC-IKVAV (SEQ ID NO: 23) group; *P<0.05, ns (not significant), using One-way ANOVA test.

As shown in FIG. 12 AADC-IKVAV (SEQ ID NO: 23) had no effect on the number of mobile episodes, or counter-clockwise rotations, but interestingly we did find significant effect on clockwise rotations (P=0.0128 and P=0.0181 for WT group vs full RAID treatment, and FUS-alone vs FUS+AADC-IKVAV (SEQ ID NO: 23) treatment, respectively; One-way ANOVA with Tukey's HSD test). That is, FIG. 12 shows no significant effects in tests (e.g., mobile episodes, counterclockwise rotations, clockwise rotations) for the FUS-alone treatment compared to the wild-type mice. As such, these results support that RAID can control behavior following the L-DOPA administration. Without wishing to be bound by theory, the results are consistent with the expected mechanism of action of dopamine delivered unilaterally to the CPu.

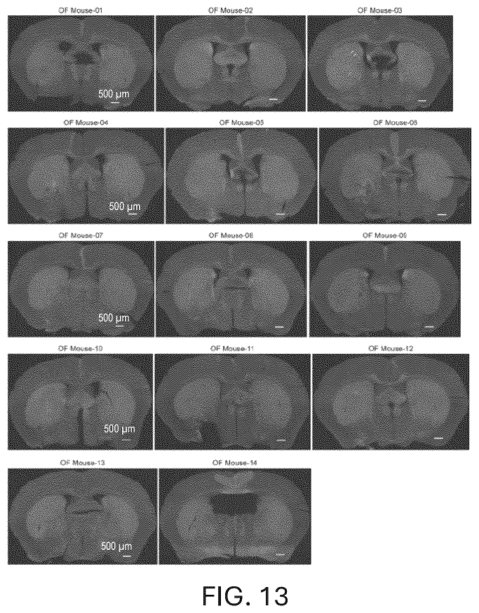

Immediately after the behavioral testing, the mice were perfused and the brains were extracted for histological analysis. Immunostaining against the delivered enzyme showed that AADC-IKVAV (SEQ ID NO: 23) was present in the FUS-targeted area in all mice treated with RAID as shown in FIG. 13 and FIG. 14. FIG. 13 illustrates representative immunostaining images of AADC staining for all mice with AADC-IKVAV (SEQ ID NO: 23) delivery related to FIG. 11. Representative immunostaining images of coronal sections from mice treated with FUS+AADC-IKVAV (SEQ ID NO: 23)+L-DOPA after behavior tests, depicting AADC staining, are displayed. The representative images of brain sections highlight the highest AADC staining signals around the striatum area in each mouse. The scale bar corresponds to 500 μm. As shown. white arrows are used to indicate AADC-IKVAV (SEQ ID NO: 23) retention in OF Mouse-03 for ease of identification.

FIG. 14 illustrates magnification of representative immunostaining images of a coronal section from a mouse treated with FUS+AADC-IKVAV (SEQ ID NO: 23)+L-DOPA for AADC staining. One or more rectangular regions (center image) within both the FUS target and contralateral site were captured at a higher magnification (left image and right image) to compare AADC+ pixel counts. The scale bars correspond to 500 μm (for the full coronal section, center image) and 100 μm (for the enlarged view, left image and right image).

FIG. 11e illustrates a graph directed to quantification of the AADC+ pixels in the FUS-targeted region (located at the left CPu) among one or more different groups, in accordance with embodiments of the present disclosure. Data is presented as mean±s.e.m. n=12 mice for wild-type+L-DOPA group, n=11 mice for FUS+L-DOPA group and n=14 mice for FUS+AADC-IKVAV (SEQ ID NO: 23)+L-DOPA group; ***P<0.001, ****P<0.0001, ns (not significant), using One-way ANOVA with Tukey's honestly significant difference test. As shown in FIG. 11e, an average of 93.3(±19.5)-fold and 13.3 (±2.8)-fold higher levels of local AADC compared to the untreated and FUS alone groups, respectively is achieved (n=12 mice for wild-type group, n=11 mice for FUS alone group and n=14 mice for FUS+AADC-IKVAV (SEQ ID NO: 23)+L-DOPA group; P<0.0001 and P=0.0002 respectively, One-way ANOVA with Tukey's HSD test).

FIG. 11f illustrates representative immunostaining images of a coronal section adjacent to an area with the highest AADC-IKVAV (SEQ ID NO: 23) retention in a mouse treated with FUS+AADC-IKVAV (SEQ ID NO: 23)+L-DOPA including rectangular areas within the FUS target at the CPu and the contralateral site magnified to compare c-Fos+ cell counts, in accordance with embodiments of the present disclosure. As shown by FIG. 11f, RAID-treated mice exhibited a 5.7(±1.8)-fold increase in ipsilateral c-Fos-positive cells (n=14 mice, P=0.0004, Two-way ANOVA with Sidak's multiple comparison test), whereas no significant changes were observed in untreated mice or mice treated with FUS alone (n=12 mice for wild-type group, n=11 mice for FUS alone group; P=0.9998 and P>0.9999 respectively, Two-way ANOVA with Sidak's multiple comparison test). FIG. 11g illustrates a graph including data directed to quantification of c-Fos+ cells in the FUS targeted region at the CPu, compared with contralateral site, in accordance with embodiments of the present disclosure. Data is presented as mean±s.e.m. n=12 mice for wild-type+L-DOPA group, n=11 mice for FUS+L-DOPA group and n=14 mice for FUS+AADC-IKVAV (SEQ ID NO: 23)+L-DOPA group; ***P<0.001, ns (not significant), using Two-way ANOVA with Sidak's multiple comparison test.

It should be noted, data presented in FIG. 11 suggests that RAID can be effective in demonstrating control of specific behaviors and induce site-specific brain activity. Thus, RAID may be a useful non-surgical method for investigating behavioral effects of localized drug action.

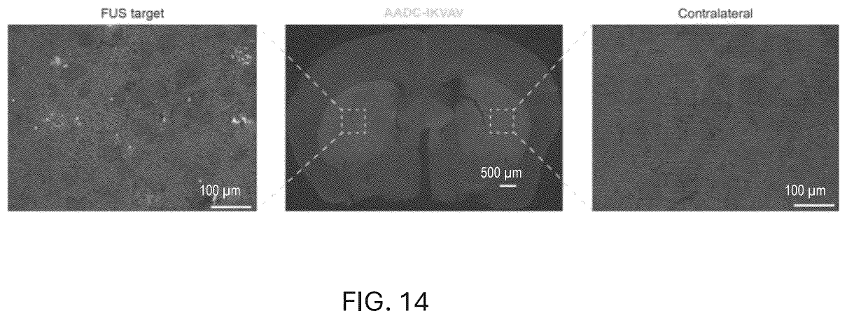

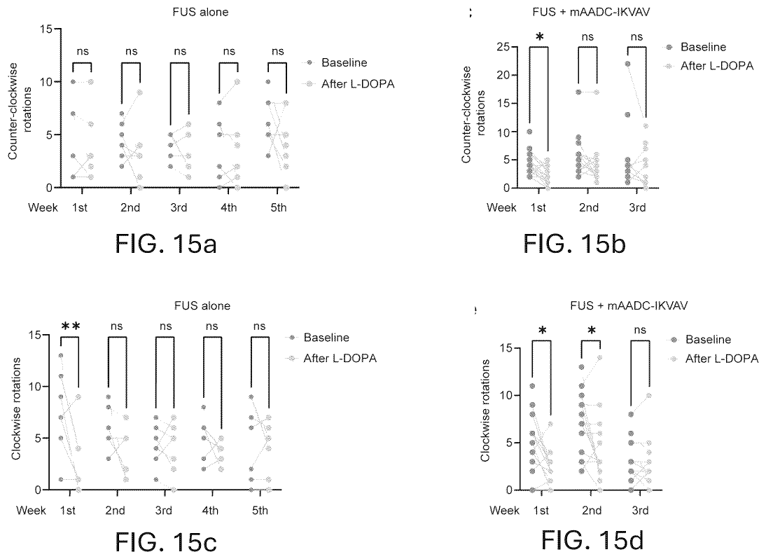

FIG. 15 includes data related to protein delivery and behavioral testing protocol repeated weekly over a course of three weeks. FIG. 15a illustrates weekly RAID-induced behavioral modulation including a first counterclockwise rotations graph measured in the open field test before and after administration of L-DOPA. FIG. 15b illustrates weekly RAID-induced behavioral modulation including a second counterclockwise rotations graph measured in the open field test before and after administration of L-DOPA. FIG. 15c illustrates weekly RAID-induced behavioral modulation including a first clockwise rotations graph measured in the open field test before and after administration of L-DOPA. FIG. 15d illustrates weekly RAID-induced behavioral modulation including a second clockwise rotations graph measured in the open field test before and after administration of L-DOPA. Data is presented as mean±s.e.m. n=7 mice for FUS alone group and n=14, 13, and 11 mice for the FUS+mAADC-IKVAV (SEQ ID NO: 23) group in weeks 1, 2, and 3, respectively; *P<0.05, **P<0.01, ns (not significant), using Two-way ANOVA with Sidak's multiple comparison test.

As shown in FIG. 15a and FIG. 15b, in the first week, consistent with a single RAID treatment, a significant reduction in counter-clockwise (ipsilateral) rotations following L-DOPA administration in mice receiving the full RAID treatment, with a reduction of 42±17% compared to baseline (n=14 mice; P=0.0323, Two-way ANOVA with Sidak's multiple comparison test). Such effect was not observed in control mice receiving FUS alone (n=7 mice; P=0.5758, Two-way ANOVA with Sidak's multiple comparison test). Interestingly, clockwise (contralateral) rotations demonstrated a different pattern. As shown in FIG. 15c and FIG. 15d, both the RAID-treated and FUS-alone groups showed a significant reduction in clockwise rotations after L-DOPA administration, by 41±17% and 53±18%, respectively (n=14 mice for RAID-treated group and n=7 mice for FUS alone group; P=0.013 and P=0.0024 respectively, Two-way ANOVA with Sidak's multiple comparison test).

As shown in FIG. 15, starting in the second week, the behavioral patterns began to shift, potentially due to behavioral adaptation. By week two, only the RAID-treated mice exhibited a reduction in clockwise rotations, with a decrease of 25±19% as shown by FIG. 15d (n=13 mice; P=0.0315, Two-way ANOVA with Sidak's multiple comparison test). FIG. 15c shows no significant effects were observed in the FUS-alone control group during this period (n=7 mice; P=0.5111, Two-way ANOVA with Sidak's multiple comparison test). It should be noted, data presented in FIG. 15 supports repeated delivery of mAADC-IKVAV (SEQ ID NO: 23) that have reorganized dopamine signaling pathways 1-4, leading to cellular adaptation and a reversal of the behavioral changes, shifting from reduced counterclockwise rotations in week one to decreased clockwise rotations in week two.

### Safety Evaluation

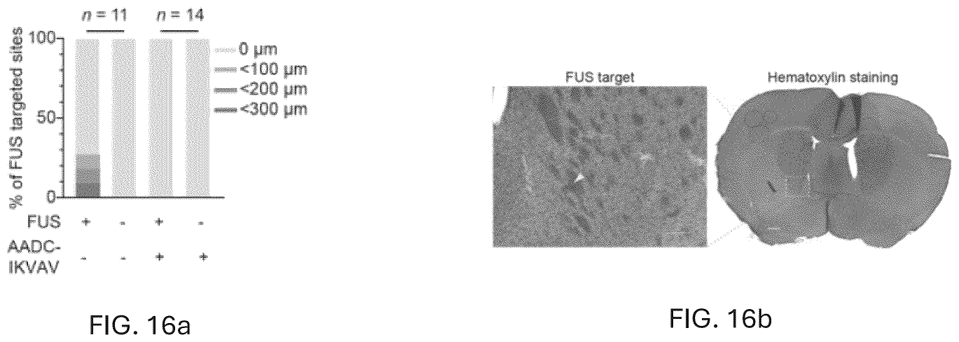

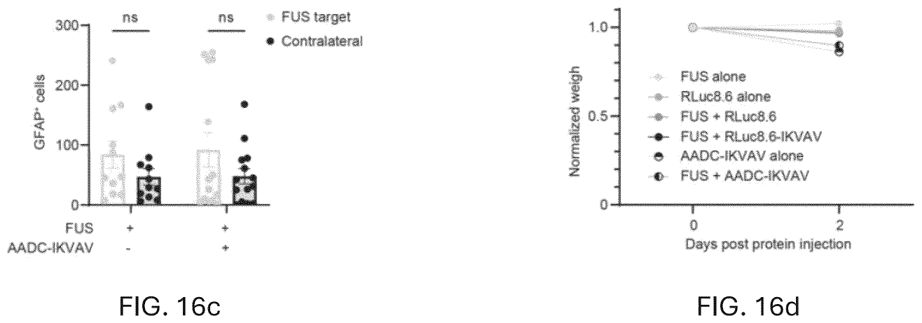

In some embodiments, to assess the safety of the RAID approach, brain sections of the mice are stained in behavior study with hematoxylin and anti-GFAP (glial fibrillary acidic protein) antibody respectively. Hematoxylin staining demonstrated that the RAID approach didn't cause observable tissue damage or bleeding as shown in FIG. 16a. FIG. 16a illustrates embodiments directed to safety assessment of RAID protocol in behavior analysis including a histological classification of brain tissue damage at 25 FUS targeted sites in 25 mice following FUS-BBBO with or without AADC-IKVAV (SEQ ID NO: 23), in accordance with embodiments of the present disclosure. The adjacent sections of those used for c-Fos staining (as shown in FIG. 11g) were used for hematoxylin staining. n=11 mice for FUS alone group and n=14 mice for FUS+AADC-IKVAV (SEQ ID NO: 23) group. Out of 25 mice treated with FUS only three (e.g., 12%) showed any detectable tissue damage. In the three mice, the damage was scattered as shown by FIG. 16b, and any damage present was contained within the area of radius between 100 and 300 μm, in line with previous reports. FIG. 16b illustrates images of representative hematoxylin-stained brain section containing a small area of hemorrhage (indicated by a white arrow) in a mouse treated with FUS alone, in accordance with embodiments of the present disclosure.

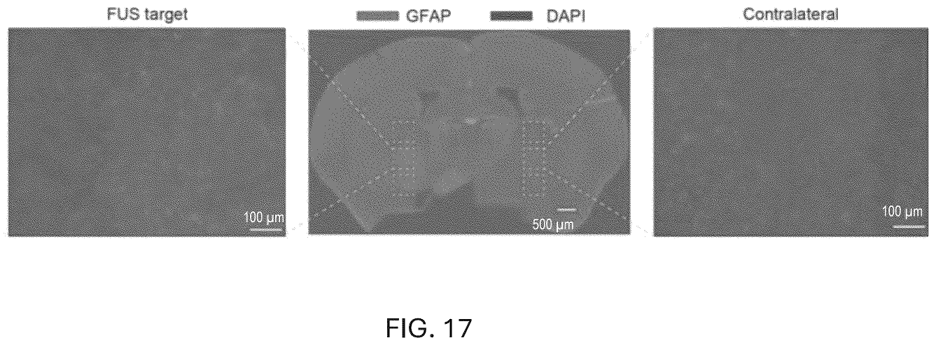

FIG. 16c illustrates a graph directed towards quantification of GFAP+ cells in the FUS targeted region at striatum, compared with contralateral site, in accordance with embodiments of the present disclosure. Data is presented as mean±s.e.m. n=11 mice for FUS alone group and n=14 mice for FUS+AADC-IKVAV (SEQ ID NO: 23) group; ns (not significant), using Two-way ANOVA test. In some embodiments, results from GFAP staining indicated that there were no significant changes in GFAP⁺ astrocytes observed in the ipsilateral region following FUS-BBBO procedure alone as shown by FIG. 16c. (n=11 mice, P=0.2328, Two-way ANOVA with Sidak's multiple comparison test;). The RAID treatment, similarly, did not affect the numbers of GFAP+ astrocytes in the targeted area when compared to the contralateral site as shown by FIG. 16c and FIG. 17 (n=14 mice, P=0.0828, Two-way ANOVA with Sidak's multiple comparison test).

FIG. 17 illustrates representative immunostaining images for GFAP in a mouse treated with FUS+AADC-IKVAV (SEQ ID NO: 23)+L-DOPA. Cell nuclei were stained with DAPI. The middle image illustrates fluorescence imaging of coronal section. The left image and the right image illustrate a magnified view of FUS targeted region (left) and contralateral site (right) of the middle image. Scale bars correspond to 500 μm (middle image) and 100 μm (left image, right image), respectively.

FIG. 16d is illustrative of a mouse body weight analysis including a weight just before FUS-BBBO or intravenous injection of unmodified or engineered RLuc8.6 and AADC-IKVAV (SEQ ID NO: 23) used to normalize the weight of each mouse during the experiment, in accordance with embodiments of the present disclosure. Data is presented as mean±s.d. n=11 mice for FUS alone group, n=10 mice per unmodified or engineered RLuc8.6 group, n=6 mice for AADC-IKVAV (SEQ ID NO: 23) alone group and n=14 mice for FUS+AADC-IKVAV (SEQ ID NO: 23) group. Intra-group differences between day 0 and 2 days after protein injection were identified using the Two-way ANOVA with Sidak's multiple comparison test. Statistical results are summarized in Table 1 included below. The Statistical significance was denoted as *P<0.05, **P<0.01, ****P<0.0001, and ns (not significant), using Two-way ANOVA test in Table 1.

| Group | Number of mice | Weight loss | P value | Significance |
|---|---|---|---|---|
| FUS alone | 11 | −2.3 ± 1.7% | 0.0974 | ns |
| RLuc8.6 alone | 10 | 2.1 ± 0.6% | 0.2148 | ns |
| FUS + RLuc8.6 | 10 | 3.4 ± 0.7% | 0.0071 | ** |
| FUS + RLuc8.6- | 10 | 3.2 ± 0.8% | 0.0118 | * |
| IKVAV (SEQ ID NO: 23) | | | | |
| AADC-IKVAV (SEQ | 6 | 13.8 ± 0.8% | <0.0001 | **** |
| ID NO: 23) alone | | | | |
| FUS + AADC- | 14 | 10.3 ± 0.5% | <0.0001 | **** |
| IKVAV (SEQ ID NO: 23) | | | | |

Table 1. The statistical results of intra-group differences between day 0 and 2 days after systemic administration of unmodified or engineered RLuc8.6 and AADC-IKVAV (SEQ ID NO: 23).

In some embodiments, throughout a duration of the RAID protocol, an analysis of the body weight of mice was conducted. A group treated with FUS+RLuc8.6 and FUS+RLuc8.6-IKVAV (SEQ ID NO: 23) experienced a weight loss of 3.4±0.7% and 3.2±0.8% respectively after 2 days following protein injection, as demonstrated by FIG. 16d and Table 1 (n=10 mice for each group, P=0.0071 and P=0.0118 respectively, Two-way ANOVA with Sidak's multiple comparison test).

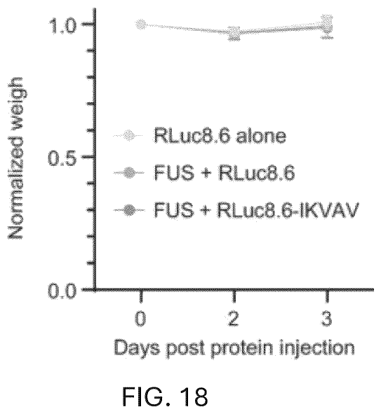

It should be noted, the observed minor weight loss was temporary, and mice began to regain body weight three days after protein injection, with no significant weight loss was observed on day 3 compared to day 0 as illustrated by FIG. 18 and Table 2. (n=5 mice for both FUS+RLuc8.6 and FUS+RLuc8.6-IKVAV (SEQ ID NO: 23) groups, with P-values of 0.9521 and 0.6775, respectively, according to the Two-way ANOVA with Sidak's multiple comparison test). FIG. 18 includes a graph directed to mouse body weight analysis after systemic administration of unmodified or engineered RLuc8.6. As shown, the weight just before intravenous injection of unmodified or engineered RLuc8.6 was used to normalize the weight of each mouse during the experiment. Data is presented as mean±s.d. n=5 mice for each group. Intra-group differences between day 0 and 3 days after protein injection were identified using the Two-way ANOVA test. The statistical results are summarized in Table 2, shown below. Statistical significance was denoted as ns (not significant), using Two-way ANOVA test in Table 2.

TABLE 2
The statistical results of intra-group
differences between day 0 and 3 days after
systemic administration of
unmodified or engineered RLuc8.6.

| | Number | | |
|---|---|---|---|
| Group | of mice | P value | Significance |
| RLuc8.6 alone | 5 | 0.9043 | ns |
| FUS + RLuc8.6 | 5 | 0.9521 | ns |
| FUS + RLuc8.6- | 5 | 0.6775 | ns |
| IKVAV | | | |
| (SEQ ID NO: 23) | | | |

In some embodiments, as shown in FIG. 16d and Table 1, a systemic injection of AADC-IKVAV (SEQ ID NO: 23) exhibited a significant and larger decrease in body weight of 10.3±0.5% within 2 days of FUS-BBBO (n=14 mice, P<0.0001, Two-way ANOVA with Sidak's multiple comparison test). In contrast, mice subjected to FUS alone did not display any notable changes in body weight (n=11 mice, P=0.0974, Two-way ANOVA with Sidak's multiple comparison test). In certain embodiments, a separate control group of mice, were administered AADC-IKVAV (SEQ ID NO: 23) intravenously without utilizing FUS to evaluate peripheral effects of the AADC enzyme, resulting in a considerable reduction in body weight of 13.8±0.8% within 2 days post-injection (n=6 mice, P<0.0001, Two-way ANOVA with Sidak's multiple comparison test). Overall, the RAID approach showed no tissue damage or bleeding, and no significant changes in astrocytes, indicating safety to the brain tissue. The weight loss, while present, was associated with systemic administration of AADC and its peripheral effects that may be suppressed with non-BBB-permeable carbidopa.

### Gene Delivery-Enabled RAID Therapeutics

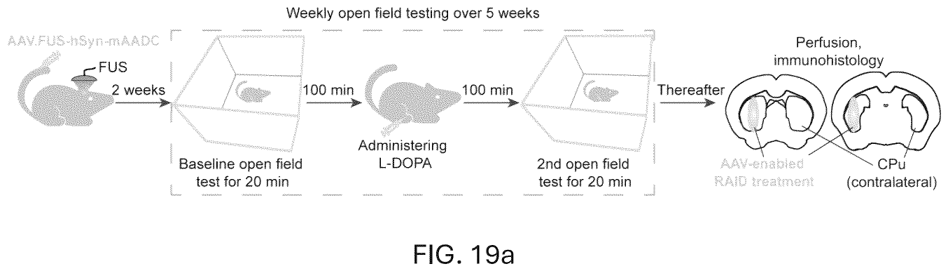

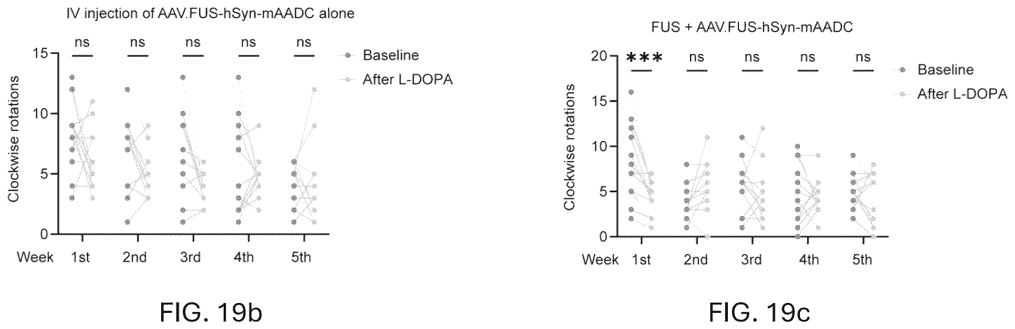

In some embodiments, a versatility and durability of RAID neuromodulation may be improved by transitioning to a gene-based approach. By encoding the RAID enzyme directly into cells via gene delivery, RAID can enable sustained, endogenous production of the enzyme, thereby extending the therapeutic window and reducing the need for repeated protein administrations. This gene-based strategy leverages the advantages of prolonged expression and precise spatial control, offering new possibilities for long-term and adaptable neuromodulation. To demonstrate long-term and adaptable neuromodulation, FUS-BBBO can be employed to noninvasively deliver the AADC gene, packaged in an engineered AAV specifically designed for local neuronal transduction at the FUS target site. The gene is delivered to two sites in the left striatum of wild-type mice, followed by a two-week period for intracellular gene expression. Locomotor activity was assessed using the open field (OF) test, first for baseline activity over 20 minutes and then again after an intraperitoneal injection of L-DOPA (2 mg/kg), with a 100-minute interval between the tests as shown by FIG. 19a.

FIG. 19a is directed to gene delivery-driven RAID modulation of behavior including an experimental scheme for AAV-driven RAID modulation of motor behavior through targeted delivery of mAADC to striatal neurons, in accordance with embodiments of the present disclosure. In some embodiments, FUS may be used to deliver the engineered AAV, termed AAV.FUS, for acoustically targeted gene delivery of mAADC (referred to as AAV.FUS-hSyn-mAADC) to two sites in the left striatum or through an i.v. injection of an AAV control. mAADC is expressed intracellularly under the control of the hSyn promoter. After 2 weeks of gene expression, an open field test (OFT) was performed twice-before and after a single injection of the prodrug L-DOPA (2 mg/kg, i.p.)—with a 200-minute interval between tests. Locomotor activity was recorded for 15 minutes following a 5-minute habituation period in each test. This L-DOPA administration and behavioral testing procedure was repeated weekly for 5 weeks. After the final behavioral test, mice were perfused 2 hours after L-DOPA administration for histological examination of mAADC gene expression and neuron activation (c-Fos+) to confirm localized neuromodulation.

FIG. 19b illustrates a clockwise rotation in mice treated with an i.v. injection of AAV control measured in the open field test before and after L-DOPA administration, in accordance with embodiments of the present disclosure. FIG. 19c illustrates a clockwise rotation in mice treated with an i.v. injection of FUS+AAV control measured in the open field test before and after L-DOPA administration, in accordance with embodiments of the present disclosure. Data is presented as mean±s.e.m. n=12 mice for each group; ***P<0.001, ns (not significant), using Two-way ANOVA with Sidak's multiple comparison test.

In the first week, mice receiving FUS-mediated AAV delivery encoding intracellular mAADC exhibited a significant reduction in clockwise (ipsilateral) rotations following L-DOPA administration, with a reduction of 43±6% compared to baseline as shown in FIG. 19b and FIG. 8c (n=12 mice; P=0.0005, Two-way ANOVA with Sidak's multiple comparison test). In contrast, control mice receiving intravenous AAV injections without FUS-BBBO showed no significant changes (n=12 mice; P=0.3076, Two-way ANOVA with Sidak's multiple comparison test). Although this effect diminished in subsequent weeks, potentially due to behavioral adaptation, the results highlight the potential of gene delivery to enable RAID-mediated neuromodulation.

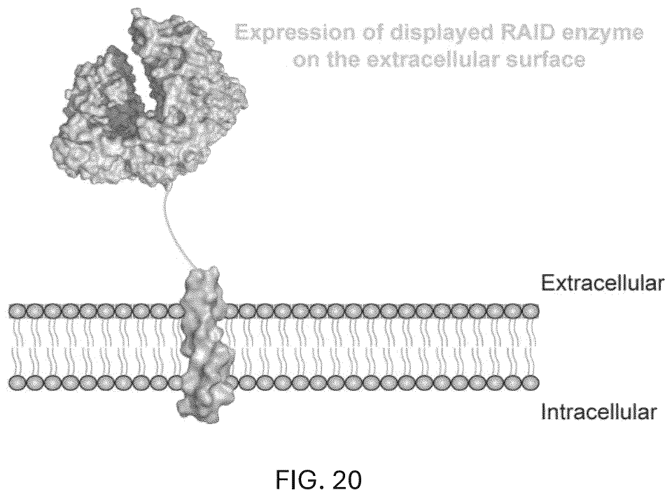

FIG. 20 illustrates gene delivery-driven expression of the displayed RAID enzyme on the extracellular surface. Further optimization by displaying the RAID enzyme on the cell surface as shown by FIG. 20. Extracellular localization enhances prodrug conversion efficiency and enable faster action on cell-surface targets, further expanding the therapeutic potential of RAID.

In the systems and methods (e.g., RAID, RAID concept, RAID strategy) described herein, a noninvasive, non-genetic, site-specific neuromodulation, was demonstrated. RAID can use FUS-BBBO to deliver an engineered enzyme that attaches to the brain interstitium and equips the targeted site with ability to produce drugs from BBB-permeable, systemically supplied inert prodrugs. RAID has unique advantages that may be used to study brain activity or in therapy planning. First, RAID is non-genetic, thus reducing the concerns of immunogenicity of viral vectors and trouble with their re-administration. Second, RAID uses delivery of proteins with FUS-BBBO, which can be delivered using safe ultrasound pressures, with protein enzymes being well below the limit of the particle size that can be delivered with FUS-BBBO. Third, RAID allows for neuromodulation to take place even the BBB is intact, unlike in the case FUS-BBBO-based delivery of small molecule drugs where drug presence is inherently linked with the opened BBB presenting a confounding factor to neuromodulation. RAID allows for a single FUS-BBBO procedure to provide multi-day long neuromodulation, as opposed to several hours while the BBB stays open, with further improvement to the RAID enzymes potentially extending this timeline. Additionally, unlike in the case of theoretical long-term drug release from nanoparticles, RAID is designed to tune the magnitude of neuromodulation in the brain by simply changing the dose of the prodrug. Similarly, by increasing the dose of the prodrug, one can compensate for the loss of enzyme over time, providing stable degree of drug activation over time.

RAID can induce changes in localized neuronal activity, and to modulate behavior with a specific pair of an enzyme and prodrug. However, RAID is a versatile concept. RAID may be compatible with any enzyme that can turn a prodrug into an active drug and be delivered to the brain with FUS-BBBO and effect on-demand localized drug action. Disclosed herein are capabilities of RAID in mice with localized delivery and multi-day retention of activity for two example enzymes.

A single injection of L-DOPA significantly activated cells expressing either of the tested dopamine receptors (D1 and D2) in the vicinity of delivered AADC-IKVAV (SEQ ID NO: 23). Additionally, expected behavioral effects were observed, that were consistent with previously published work. Unexpectedly, even wild-type mice showed a degree of lateralized rotations. Such rotations are caused by the asymmetrical environment of our custom-made open field arenas (e.g., uneven light reflection, fan noise), in accordance with previous studies showing that environmental context influences open-field behavior, especially when combined with the habituation leading to lower overall movement of mice when placed in the open field the second time. Negative controls (FUS alone group) behaved indistinguishably from wild-type mice and the changes in rotation behavior induced by RAID treatments were consistent with previous studies involving unilateral intracranial dopamine injections into the GPu.

While significant effects of the AADC delivery alone were not reported on c-Fos accumulation or on two of the three covariates tested in the open field, an effect of AADC on clockwise rotations as shown in FIG. 12 is demonstrated. Without wishing to be bound by theory, one possibility is that in wild-type mice AADC convert endogenous L-DOPA into dopamine and introduce background activation of neurons in the caudate putamen, presenting an exciting possibility that AADC alone, even without prodrugs could be sufficient for site-specific therapy. However, this effect would likely be reduced in Parkinson's disease, where endogenous L-DOPA levels are lower and where AADC/L-DOPA RAID would be most useful as a therapeutic strategy. While significant effects were observed in one of the tested scenarios, it is possible that multiple factors, such as asymmetry of the rotations in the open field, residual background levels of L-DOPA, or other effects coincided in that scenario to produce the isolated significant effect. In some embodiments, if the administration of an enzyme alone can indeed produce lasting behavioral effects dependent on the background conversion of endogenous neurotransmitters, it may present a useful tool for interrogation of neural circuitry or therapy that is drug-free. Specifically, for those neurotransmitters that do not have a BBB-permeable prodrug, administration of RAID enzyme alone may provide localized neuromodulation. In the intended application of RAID as a tool for investigation of circuitry or therapy planning, one aspect offered by RAID is that induces localized neuromodulation that results in a distinguishable behavioral readout after the treatment, with or without the prodrug administration.

With that in mind, RAID may be useful in a number of scenarios including, for example, large animal studies, as a noninvasive and reversible alternative to lesioning or deep-brain stimulation. Without the need of surgical resection or viral vector administration, multiple brain regions could be studied on different days using RAID. Without genetic delivery, RAID would not result in induction of neutralizing antibodies against the viral vectors. In therapy planning, for example, RAID could be used to validate the presumed seizure focus, before resection, genetic modification for chemogenetic neuromodulation (e.g., with Acoustically Targeted Chemogenetics (ATAC)), or invasive deep brain stimulation, with use of a combination of drugs and enzymes that silence neuronal activity. An unacceptable side effect profile of such silencing, or lack of effects on seizure reduction over several-day long RAID neuromodulation could help refine the site or cell-type targeted for treatment.

Further improvements to RAID may include using other cell-adhesive peptides to improve attachment and retention of the delivered enzymes to the surrounding brain cells. Enzyme retention was enhanced through fusing with ECM-mimicking peptide that binds to neurons. This led to a 1-week long retention of RLuc8.6-IKVAV (SEQ ID NO: 23) in the brain after a single session of FUS-BBBO delivery. Additionally and/or alternatively, an approach to tether RAID enzymes to ECM, which has previously been used to capture and retain the secreted ECM proteins. Other improvements may include improving the tissue specificity of the delivered enzymes, to ensure lower exposure of peripheral tissues to the enzyme. As disclosed herein, RAID enables studying effects of localized neuromodulation.

It should be noted, analysis of RAID was conducted using AADC and L-DOPA to control dopamine receptor-expressing brain cells. One or more additional enzyme prodrug pair may be envisioned for analysis with RAID. For example, naturally occurring enzymes in the body reduce concerns of immune response against the enzyme, and use of a clinically approved drug, L-DOPA, enables a path of translation. RAID allows for prodrug-induced site-specific effects of neuromodulation, forming a good tool to plan therapy or study the effects of site-specific neuromodulation. On the other hand, AADC itself may have side effects due to its ability to synthesize other neurotransmitters (such as serotonin) and trace amine neuromodulators including phenylethylamine, tyramine, and tryptamine. AADC-IKVAV (SEQ ID NO: 23) might catalyze the above decarboxylation reactions utilizing the endogenous aromatic L-amino acid substrates after delivery through the BBB. Additionally, these biological reactions can also occur peripherally after the systemic administration of AADC-IKVAV (SEQ ID NO: 23), potentially leading to weight loss due to the significant roles played by peripheral dopamine and serotonin in metabolic regulation. Such effects may be taken into consideration and managed during any potential treatment or experiment involving this specific enzyme and prodrug pair.

To mitigate potential side effects associated with naturally occurring enzymes and neurotransmitters, the development of a more specific and orthogonal enzyme-prodrug pair enable more precise neuromodulation. Another option is a reduction of systemic exposure of RAID enzymes. For example, focused ultrasound-mediated intranasal brain drug delivery (FUSIN) could be used for site-specific enzyme delivery and be followed-up with systemic administration of a prodrug to lower exposure to an enzyme in major organs. Implementing strategies like FUSIN may enhance the targeted delivery of prodrug enzymes and minimize potential systemic side effects. As disclosed herein, RAID may be useful in therapy planning and site-specific neuromodulation to investigate brain circuitry. Further improvement of each component of RAID may enable control of different cell-types and molecular pathways with greater duration, lowered side effects, and improved specificity.

Materials and Methods—Animals—Wild-type C57BL/6J mice (12-18 weeks of age) were purchased from Jackson Laboratory and housed in a 12 h light/dark cycle and were provided with water and food ad libitum. All experiments were performed under a protocol approved by the Institutional Animal Care and Use Committee of Rice University. The immunohistochemical analysis of RLuc8.6 related to FIG. 2 involved 25% female mice and 75% males. The in vivo BLI study related to FIG. 2 included an equal number of each gender. All other experiments utilized male mice.

Plasmid construction—For constructing the recombinant protein expression plasmid of RLuc8.6, the protein coding sequence (CDS) of RLuc8.6 was amplified from pcDNA-RLuc8.6-535 (Addgene ID 87125) and subcloned into the vector pRSETb (Addgene ID 89536) with a N-terminal His-tag through Gibson assembly at the BamHI and EcoRI site. The CDSs of ECM-mimicking peptides (synthesized by GenScript) were assembled to the C-terminal of RLuc8.6 for creating the expression plasmids of engineered variants. Similarly, the expression plasmid of AADC-IKVAV (SEQ ID NO: 23) was constructed by subcloning AADC amplified from Sino Biological plasmid (HG29995-CF) and IKVAV (SEQ ID NO: 23) fragment into the above vector using the same restriction sites. Primers used for cloning are listed in Table 3 below. It should be noted, AADC-IKVAV (SEQ ID NO: 23) was first subcloned into vector pTrcHisA, but only observed low protein production in Escherichia coli. However, it serves as a template for creating the expression plasmid of AADC-IKVAV (SEQ ID NO: 23) using vector pRSETb, resulting in a better yield. CDSs, noncommercial plasmid sequences, and subcloning insertion sites are listed in Table 4 below.

Protein expression and purification—The recombinant protein was expressed in Escherichia coli and purified by Ni-affinity chromatography. For RLuc8.6 and engineered variants, Escherichia coli BL21 (DE3) cell was transformed with expression construct respectively and grown in Terrific Broth (TB) medium at 37° C. to an OD₆₀₀ of ˜0.6 before induction with 0.1 mM IPTG at 20° C. for overnight. Harvested cell pellets from 1-liter cultures were resuspended in 40 mL ice-cold lysis buffer (50 mM sodium phosphate, 300 mM NaCl, 10 mM imidazole, 10% glycerol, pH 8.0) for sonication. The supernatant after centrifugation at 17, 500 RPM, 4° C. for 45 min was loaded into the glass chromatography columns (Bio-Rad, catalog number 7372522) and incubated with Ni-NTA agarose resin (Qiagen, catalog number 30210) on ice for 1 h. The column was washed and eluted with a stepwise imidazole gradient (10 mM to 500 mM) of lysis buffer through gravity flow. Eluates were concentrated with a Corning® Spin-X® UF 20 mL centrifugal filter unit (10 kDa cutoff), and then buffer exchanged into PBS using the PD-10 desalting column. Eluted proteins were concentrated again, analyzed by SDS-PAGE, and quantified by a NanoDrop spectrophotometer. Similarly, AADC-IKVAV (SEQ ID NO: 23) was expressed in Escherichia coli BL21 (DE3) cell and purified by Ni-affinity chromatography through gravity flow as described above, except that the overexpression was induced by 0.05 mM IPTG at 22° C. and it was gradually buffer changed into PBS while being concentrated with a 30 kDa cutoff filter.

FUS-BBBO—C57BL/6J male or female mice (12-18 weeks of age) were anaesthetized with 2.5% isoflurane in 1.5% O₂ and shaved on top of skull using a trimmer. A catheter, made by a 30-gauge needle connected to PE10 tubing, was inserted into the tail vein, affixed in place using tissue glue and then flushed with 10 units (U) mL⁻¹ of heparin in sterile saline (0.9% NaCl). Subsequently, the mouse was mounted on the RK50 (FUS Instruments) stereotactic platform using the ear bars and bite bar/nose cone. A midline scalp incision was vertically made to expose the skull after disinfecting the site using three alternating scrubs of chlorhexidine scrub and chlorhexidine solution. The locations of Lambda and Bregma were registered in the RK50 software using the guild pointer. Next, sterile ultrasound gel was applied on the surface of skull before placing the ultrasound transducer in a tank, both of which were filled with degassed water. The mice were then sequentially injected via tail vein with purified recombinant protein in PBS buffer and approximately 1.5×10⁶ DEFINITY microbubbles (Lantheus) per gram of body weight diluted in sterile saline. Immediately after injections of protein and microbubbles, the mice were insonated using RK-50 FUS system with axial and lateral diameter of 5 mm and 1.2 mm, respectively. FUS target coordinates used for each experiment are listed in Table 5 shown below.

The ultrasound parameters used were 1.5 MHz, 10 ms duration, 1000 ms burst period for 120 pulses. The pressure at 0.3 MPa was used for FUS-BBBO based on preliminary tests in our lab, except that 0.36 MPa was chosen for the experiments in FIG. 2 according to previous studies. Following insonation, the mice were placed back to home cage for recovery after closing the scalp incision with tissue glue.

Immunohistochemical analysis of RLuc8.6—Mice (n=4) were injected with RLuc8.6 (20 mg/mL, 150 mg/kg, i.v.) right before FUS-BBBO targeting 4 sites at left striatum with 2 min interval between insonations. After 1 h, the mice were sacrificed by transcardial perfusion with cold heparinized (10 U mL⁻¹) PBS following induction of anesthesia using ketamine/xylazine solution (80 mg/kg and 10 mg/kg, respectively), and immediately afterwards with 10% neutral buffered formalin. The brains were extracted and postfixed for 24-48 hours in the same fixative at 4° C. before being sliced into 50 μm coronal sections using a vibratome (Leica). The slices were blocked in 10% normal goat serum (SouthernBiotech) and 0.3% Triton-X solution in PBS for 1 h at room temperature and then incubated with a primary rabbit anti-RLuc antibody (1:1000, PA1-180, ThermoFisher) in blocking buffer for overnight at 4° C. Subsequently, the sections were washed three times (15 min each) in PBS and then incubated with a secondary goat anti-rabbit antibody conjugated to Alexa Fluor 647 (1:500, A-21245, ThermoFisher) in blocking buffer for 2 h at room temperature. After washing three times (15 min each) in PBS, these sections were mounted onto glass slides using the mounting media (Vector Laboratories) with DAPI and allowed to air-dry overnight in dark prior to imaging.

In vivo BLI—Two groups of mice (n=7 mice for FUS+RLuc8.6 group and n=3 mice for FUS alone group) underwent FUS-BBBO targeting one site at left striatum immediately after i.v. injection of RLuc8.6 (2 mg/mL, 8 mg/kg) or equivalent volume of PBS buffer. The third group of mice (n=6) was injected intravenously with the same dose of RLuc8.6 without FUS-BBBO procedure. BLI was conducted 1 h, 24 h, 48 h and 96 h after FUS insonation with an IVIS spectrum imager (Perkin Elmer). The mice were injected with CTZ (2.5 mg/mL, 3.5 mg/kg, i.p.; cat #303-INJ, Nanolight Technology) after being anaesthetized with 2.5% isoflurane in 1.5% O₂. Bioluminescence images under similar anesthesia were taken every 5 mins until luminescent signal of the head peaked, usually 5-15 min after injection of CTZ. The BLI parameters used were open filter for emission, automatic exposure time (mostly 5 s), aperture (f/stop) 1, binning 8, field of view A (3.9 cm, imaging the head) and C (13 cm, imaging the whole body). The bioluminescence signal was quantified by calculating the average radiance (p/s/cm²/sr) in the head region of view C imaging using the Living Image software (Caliper Life Sciences). In the FUS+RLuc8.6 group, one mouse exhibited a 50% lower ultrasonic signal amplitude compared to the others with FUS treatment, indicating an ineffective FUS-BBBO procedure and leading to its exclusion from the analysis.

Ex vivo analysis of engineered RLuc8.6—FUS was performed to target three sites in the left hemisphere of C57BL6J mice (n=5 mice per group) immediately after systemic administration of RLuc8.6 (20 mg/mL, 100 mg/kg) or engineered variant (20 mg/mL, 104 mg/kg) and microbubbles by tail vein injection. The dose of engineered RLuc8.6 was increased accordingly based on their molecular weight for injecting the same number of molecules as unmodified RLuc8.6. The control group (n=5 mice) was injected intravenously with the same dose of unmodified RLuc8.6 without FUS. The mice were euthanized using CO₂ without perfusion at two time points (2-day and 7-day) after FUS-BBBO procedure. Immediately afterwards, the brains were extracted, washed with ˜20 mL PBS buffer in a 50 mL conical tube for 30 s, and then cut into 2 mm sections without olfactory bulb and brainstem using a coronal Slicer (Invitrogen). The sections were individually transferred into a 6-well glass bottom plate (Cellvis) filled with 2 mL PBS buffer. BLI was performed using an IVIS spectrum imager (Perkin Elmer) immediately after adding 1 mL dissolved CTZ (cat #303-INJ, Nanolight Technology) with a final concentration of 10 μM. The parameters used here were similar as in vivo BLI with is of exposure time and field of view C. The average radiance (p/s/cm²/sr) of each brain section was quantified with the Living Image software (Caliper Life Sciences) and summed to compare the activity retention of RLuc8.6 among different groups. At timepoint of 2 days, two mice (each from RLuc-8.6-YIGSR (SEQ ID NO: 25) and RLuc8.6-GRGDS (SEQ ID NO: 24) group respectively) showed FUS-BBBO-related tissue damage, resulting abnormally high levels of bioluminescence, and thus were excluded from analysis.

c-Fos activation with engineered AADC—Three groups of mice underwent FUS-BBBO procedure to target a single site at left striatum immediately after intravenous injection of recombinant protein AADC-IKVAV (SEQ ID NO: 23) (20 mg/mL, 250 mg/kg) or equivalent volume of PBS buffer (n=5 mice for FUS alone+L-DOPA group). After 48 h, the mice were given a single dose of L-DOPA (0.1 mg/mL, 2 mg/kg, i.p.; Spectrum Chemical) 10 min after injection of carbidopa (1 mg/mL, 25 mg/kg, i.p.; Sigma-Aldrich), both of which were dissolved in sterile saline containing 2.5 and 0.125 mg/mL ascorbic acid. The FUS+AADC-IKVAV (SEQ ID NO: 23) control group (n=6 mice) did not receive L-DOPA injection, while the experimental group (n=6 mice for the FUS+AADC-IKVAV (SEQ ID NO: 23)+L-DOPA group) received L-DOPA injection. After 2 hours, the mice were euthanized by transcardial perfusion, and their brains were extracted and sliced into 50 μm coronal sections following 24-48 hours of fixation.

Immunostaining of AADC was performed as follows: (1) incubate the brain sections in 1× antigen retrieval solution (catalog number: 00-4955-58; Invitrogen) overnight in a 60° C. water bath; (2) block sections in 5% normal goat serum (SouthernBiotech) and 0.3% Triton-X solution in PBS for 1 h at room temperature; (3) incubate with primary rabbit anti-AADC antibody (1:500, 10166-1-AP, Proteintech) for staining AADC-IKVAV (SEQ ID NO: 23) in blocking buffer for 2 h at room temperature; (4) after washing three times in PBS (15 min each, the same as below), incubate with secondary goat anti-rabbit antibody conjugated to Alexa Fluor 647 (1:750, A-21245, ThermoFisher) in blocking buffer for 2 h at room temperature; (5) after washing three times, mount sections onto glass slides using the mounting media (Vector Laboratories) with DAPI and air-dry overnight in dark before imaging.

The section displaying the strongest AADC fluorescence was selected as the representative for each mouse, and its adjacent section was stained separately to identify c-Fos and dopamine receptor D1 and D2 positive cells. The procedure was carried out as follows: (1) block sections in 10% normal goat serum (SouthernBiotech) and 0.3% Triton-X solution in PBS for 1 h at room temperature; (2) incubate with primary rabbit anti-c-Fos (1:2000, 2250S, Cell Signaling Technology) antibody in PBS with 0.3% Triton-X for 2 h at room temperature; (3) after washing three times in PBS (15 min each, the same as below), incubate with primary rat anti-D1 Dopamine Receptor (1:500, D2944, MilliporeSigma) and Guinea pig anti-Dopamine Receptor D2 (1:500, Cat. #376 205, Synaptic Systems) antibodies in 5% normal goat serum and 0.3% Triton-X solution in PBS overnight at 4° C.; (4) after washing three times, incubate with secondary goat anti-rabbit antibody conjugated to Alexa Fluor 647 (1:500, A-21245, ThermoFisher) in PBS with 0.3% Triton-X for 2 h at room temperature; (5) after washing three times, incubate with secondary goat anti-rat antibody conjugated to Alexa Fluor 546 (1:500, A-11081, ThermoFisher) and goat anti-Guinea Pig antibody conjugated to Alexa Fluor 488 (1:500, A-11073, ThermoFisher) in 5% normal goat serum and 0.3% Triton-X solution in PBS for 2 h at room temperature; (6) after washing three times, mount sections onto glass slides using the mounting media (Vector Laboratories) with DAPI and air-dry overnight in dark before imaging.

Histological imaging—All histological images were obtained using a fluorescence microscope (BZ-X810, Keyence). To evaluate RLuc8.6 retention, as depicted in FIG. 2b and FIG. 3, we acquired fluorescence images of striatal sections using a ×4 objective in DAPI (nuclei) and far-red (RLuc8.6) channels. Subsequently, we identified a 3×3 binned area within the FUS target region of the representative section in the left striatum, exhibiting the highest RLuc8.6 retention. This specific area was then imaged using a ×40 objective, while also capturing images of the corresponding contralateral areas in the right striatum.

To analyze AADC-IKVAV (SEQ ID NO: 23) retention, as depicted in FIG. 7 and FIG. 11, stained coronal sections within the striatum were captured using a ×4 objective in DAPI (nuclei, not shown) and far-red (AADC) channels. Among these sections, the one displaying the most intense AADC fluorescence was chosen as the representative for each mouse. Subsequently, the selected sections were further examined at a higher magnification (×40 objective) for quantitative comparison. Specifically, we identified a rectangular area (5×9 binning each) within the FUS target region in the left striatum of each representative section, which exhibited the highest AADC-IKVAV (SEQ ID NO: 23) retention. This area was imaged using a ×40 objective. Additionally, corresponding contralateral areas in the right striatum were also imaged.

To quantify the c-Fos-positive cells as shown in FIG. 7, stained coronal sections were initially imaged using a ×4 objective in DAPI (nuclei, not shown), Cy3 (D1R), green (D2R), and far-red (c-Fos) channels. Within the left striatum region, a specific 5×9 binned rectangle area was selected corresponding to the highest observed AADC fluorescence in the adjacent AADC-stained section. Subsequently, images were captured of these areas using a 40× objective. The corresponding contralateral areas in the right striatum were also imaged.

To quantify the c-Fos-positive cells related to FIG. 11, the stained coronal sections were initially imaged using a 4× objective in DAPI (nuclei, not shown) and far-red (c-Fos, shown in purple) channels. Within the left striatum region, one or more specific 3×3 binned square areas were selected corresponding to the highest observed AADC fluorescence in the adjacent AADC-stained section. Subsequently, images of these areas were captured using a 40× objective. Corresponding contralateral areas in the right striatum were also captured.

To assess the safety of the RAID protocol, as shown in FIG. 15, the hematoxylin-stained brain sections were initially imaged using only the brightfield channel with a 4× objective. Subsequently, sections exhibiting hemorrhage were further observed at a higher magnification (40× objective). Additionally, the brain sections stained for GFAP were imaged using a 4× objective in DAPI (nuclei) and far-red (GFAP) channels. Three square areas were then selected in both the left and right striatum respectively, each with 3×3 binning, for imaging with a 40× objective to quantify GFAP+ cells.

Quantitative analysis of histology images—To quantify the c-Fos positive cells, as shown in FIGS. 4 and 5, the histological images were analyzed using ZEISS ZEN software (Version 3.5). In FIG. 7, we applied the following brightness (white) thresholds for the corresponding channels: 6045 (DAPI, nuclei, not shown), 15,000 (Cy3, D1R), 16,000 (green, D2R), and 6045 (far-red, c-Fos), with a gamma value of 1. In FIG. 11, we applied a Brightness (White) threshold of 6045 for both DAPI (nuclei, not shown) and far-red (c-Fos) channels, with a Gamma value of 1. The images were then overlayed, and the c-Fos+ cells were manually counted. Similarly, the GFAP+ cells were manually counted, as shown in FIG. 15c and FIG. 17, by applying a Brightness (White) threshold of 5686 and a Gamma value of 1 for both the DAPI (nuclei) and far-red (GFAP, purple) channels.

To quantify the AADC-IKVAV (SEQ ID NO: 23) retention in the FUS targeted region, as shown in FIG. 7 and FIG. 11, the histological images were analyzed using ImageJ (version 1.53t). Specifically, the average pixel intensity of the AADC-IKVAV (SEQ ID NO: 23) channel image captured on the contralateral side of each representative section was calculated to establish the background for that section. Subsequently, the number of positive pixels of the image captured on the FUS target side of that section was determined, defined as pixels exhibiting an intensity value more than 3-fold higher than the background observed in the corresponding contralateral side image.

To manually count the c-Fos-positive cells in FIG. 7, three scorers (Z. H., S. N., and R. J.) independently assessed all c-Fos staining sites. R. J. served as the blinded scorer, and the averaged counts from all three scorers were rounded for the final statistical analysis.

To address potential bias and validate our counting approach, all c-Fos staining sites from mice in FIG. 11 were independently counted by 4 scorers (A. M., Z. H., C. H. and R. J.). C. H. acted as the blinded scorer. The comparisons among different groups yielded consistent trends. For instance, RAID-treated mice showed a 5.7(11.8)-fold increase (counted by A. M. and Z. H.) and a 9.2(12.4)-fold increase (counted by C. H. and R. J.) in ipsilateral c-Fos-positive cells compared to the contralateral site in the behavior study (n=14 mice, P=0.0004 and P=0.0001, respectively, Two-way ANOVA with Sidak's multiple comparison test). The inter-experimenter variability (average percentage difference) for the FUS+AADC-IKVAV (SEQ ID NO: 23)+L-DOPA group was 15.1±5% for the FUS target (n=14 mice, P=0.468, paired two-tailed t-test) and 26.7±7.9% for the contralateral site (n=14 mice, P=0.0585, paired two-tailed t-test).

Locomotor behavior test—Two groups of mice underwent FUS-BBBO targeting two sites at left striatum immediately after intravenous injection of recombinant protein AADC-IKVAV (SEQ ID NO: 23) (n=14 mice for FUS+AADC-IKVAV group, 20 mg/mL, 250 mg/kg) or equivalent volume of PBS buffer (n=11 mice for FUS alone group). Another group without FUS-BBBO procedure served as wild type control (n=12 mice for WT group). After 46 h, the first session of behavioral test was performed in a custom-made non-transparent open field box (30.5 cm×30.5 cm) as a baseline. Each mouse was individually placed into the apparatus center with a light intensity and background noise at ˜320 lux and 46 dB (400 Hz peak), respectively. After a habituation period of 5 min, the locomotor activity of a freely-moving mouse was recorded for 15 min by a video tracking system (Stoelting Co.) consisting of an overhead camera connected to a computer with Any-Maze software. Behavioral measures include total distance traveled, average and maximum speed, freezing time and episodes, mobile/immobile time and episodes, clockwise and counterclockwise rotations and head turn angles. The mouse was returned to the home cage immediately after testing. After 90 min, the mice were intraperitoneally injected with carbidopa (25 mg/kg) and 10 min later L-DOPA (2 mg/kg) as described above. A second session of open field test 100 min after L-DOPA injection.

Immediately afterwards, the mice were sacrificed for immunohistochemical analysis of AADC-IKVAV (SEQ ID NO: 23) retention as already mentioned. The section displaying the strongest AADC fluorescence was selected as the representative for each mouse, and its adjacent section was stained separately to identify c-Fos positive cells. The procedure was carried out as follows: (1) block sections in 10% normal goat serum (SouthernBiotech) and 0.3% Triton-X solution in PBS for 1 h at room temperature; (2) incubate with primary rabbit anti-c-Fos (1:2000, 2250S, Cell Signaling Technology) and chicken anti-tyrosine hydroxylase (1:2000, SKU: TYH, Aves Labs) antibodies in PBS with 0.3% Triton-X for 2 h at room temperature; (3) after washing three times in PBS (15 min each, the same as below), incubate with primary mouse Anti-6X His tag antibody (1:10000, ab18184, Abcam) in blocking buffer overnight at 4° C.; (4) after washing three times, incubate with secondary goat anti-rabbit antibody conjugated to Alexa Fluor 647 (1:500, A-21245, ThermoFisher) and goat anti-chicken antibody conjugated to Alexa Fluor 488 (1:500, A-11039, ThermoFisher) in PBS with 0.3% Triton-X for 2 h at room temperature; (5) after washing three times, incubate with secondary goat anti-mouse antibody conjugated to Alexa Fluor 546 (1:1000, A-21143, ThermoFisher) in blocking buffer for 2 h at room temperature; (6) after washing three times, mount sections onto glass slides using the mounting media (Vector Laboratories) with DAPI and air-dry overnight in dark before imaging.

Safety analysis—To investigate potential lesions resulting from the use of FUS-BBBO in the RAID protocol, hematoxylin staining was performed (n=11 mice for FUS alone+L-DOPA group and n=14 mice for FUS+AADC-IKVAV (SEQ ID NO: 23)+L-DOPA group) on the adjacent sections of each representative section used for c-Fos staining, which was associated with behavior analysis as shown in FIG. 11. Hematoxylin staining was conducted according to the following steps: (1) The section was immersed in 100% ethanol for 1 minute; (2) It was then soaked in 95% ethanol for 1 minute; (3) Subsequently, a 1-minute wash in H₂O was performed; (4) The section was soaked in 50% hematoxylin (H&E Staining Kit, Abeam ab245880) for 1 minute; (5) This was followed by a 3-minute wash in H₂O; (6) A 20-second soak in Bluing Reagent (H&E Staining Kit, Abcam ab245880) was carried out; (7) Another 3-minute wash in H₂O followed; (8) Finally, the sections were mounted onto glass slides using mounting media (Vector Laboratories) and air-dried overnight in the dark before imaging.

Adjacent sections were stained separately with an anti-GFAP antibody to examine astrocytic activation. The staining procedure involved the following steps: (1) The sections were blocked in a solution of 10% normal goat serum (SouthernBiotech) and 0.3% Triton-X in PBS for 1 hour at room temperature; (2) They were then incubated with primary mouse anti-GFAP antibody conjugated to Alexa Fluor 647 (1:500, sc-33673 AF647, Santa Cruz Biotechnology) in the blocking buffer for 2 hours at room temperature; (3) Following three washes in PBS (15 minutes each), the sections were mounted onto glass slides using mounting media (Vector Laboratories) with DAPI and left to air-dry overnight in the dark before imaging.

To assess the potential impact of the RAID approach on the body weight of the mice involved in behavior analysis (n=11 mice for the FUS alone+L-DOPA group and n=14 mice for the FUS+AADC-IKVAV (SEQ ID NO: 23)+L-DOPA group), their body weight was recorded on a daily basis before and after FUS-BBBO administration. Additionally, an additional group of mice (n=6 mice) was included for comparison, which received intravenous injections of the same dose of AADC-IKVAV (SEQ ID NO: 23) (20 mg/mL, 250 mg/kg) but did not undergo any FUS-BBBO treatment. The body weight of the additional group of mice was also recorded in a similar manner; however, unintentionally four mice were omitted from weighing on the day following the AADC-IKVAV (SEQ ID NO: 23) injection. Similarly, the mouse body weight was recorded before and after the intravenous injection of unmodified or engineered RLuc8.6, which is related to the study presented in FIG. 5.

Statistical analysis—Statistical analysis was performed using GraphPad Prism software version 9.0. All quantitative data were presented as mean±s.e.m., except for normalized weight in FIG. 16d and FIG. 18, mean±s.d. A two-tailed ratio paired t-test was used for FIG. 2c. One-way ANOVA followed by Tukey's honestly significant difference test was used for comparing the means of three or more independent groups. Two-way ANOVA followed by Sidak's multiple comparison test was used to compare the mean differences between groups that are affected by two factors. The difference between groups was considered statistically significant when *p<0.05, **p<0.01, ***p<0.001, ****p<0.0001.

TABLE 3
Synthetic oligos used for plasmid construction in this study.

| | Vector | | SEQ | | Restriction |
|---|---|---|---|---|---|
| | backbone | Name | ID NO | Sequences (5′ to 3′) | sites |
| RLuc8.6 | PRSETb | RLuc8.6 F | 1 | TGTACGACGATGACGATAAGGATCCGAT | BamHI or |
| | | | | GGCTTCCAAGGTGTACGACCCCG | EcoRI sites |
| | | RLuc8.6 R | 2 | AGCAGCCGGATCAAGCTTCGAATTCTTA | are |
| | | | | CTGCTCGTTCTTCAGCACGCGCT | underlined. |
| RLuc8.6- | PRSETb | RLuc8.6 F | 3 | TGTACGACGATGACGATAAGGATCCGAT | BamHI or |
| IKVAV | | | | GGCTTCCAAGGTGTACGACCCCG | EcoRI sites |
| (SEQ ID | | RLuc8.6 R | 4 | CTGCTCGTTCTTCAGCACGCGCT | are |
| NO: 23) | | DNA | 5 | GAGCGCGTGCTGAAGAACGAGCAGGGA | underlined. |
| | | fragment of | | GGAAGTGGCAGCTCTGGCGGCAGTGGA | |
| | | IKVAV | | GGGTCTGGTGGCAGCGGAATCAAAGTC | |
| | | (SEQ ID NO: | | GCCGTGTAAGAATTCGAAGCTTGATCCG | |
| | | 23) | | GCTGCTAACAA | |
| RLuc8.6- | PRSETb | RLuc8.6 F | 6 | TGTACGACGATGACGATAAGGATCCGAT | BamHI or |
| YIGSR | | | | GGCTTCCAAGGTGTACGACCCCG | EcoRI sites |
| (SEQ ID | | RLuc8.6 R | 7 | CTGCTCGTTCTTCAGCACGCGCT | are |
| NO: 25) | | DNA | 8 | GAGCGCGTGCTGAAGAACGAGCAGGGA | underlined. |
| | | fragment of | | GGAAGTGGCAGCTCTGGCGGCAGTGGA | |
| | | YIGSR | | GGGTCTGGTGGCAGCGGATACATTGGTT | |
| | | (SEQ ID | | CCAGATAAGAATTCGAAGCTTGATCCGG | |
| | | NO: 25) | | CTGCTAACAA | |
| RLuc8.6- | PRSETb | RLuc8.6 F | 9 | TGTACGACGATGACGATAAGGATCCGAT | BamHI or |
| GRGDS | | | | GGCTTCCAAGGTGTACGACCCCG | EcoRI sites |
| (SEQ ID | | RLuc8.6 R | 10 | CTGCTCGTTCTTCAGCACGCGCT | are |
| NO: 24) | | DNA | 11 | GAGCGCGTGCTGAAGAACGAGCAGGGA | underlined. |
| | | fragment of | | GGAAGTGGCAGCTCTGGCGGCAGTGGA | |
| | | GRGDS | | GGGTCTGGTGGCAGCGGAGGTCGTGGT | |
| | | (SEQ ID | | GATAGTTAAGAATTCGAAGCTTGATCCG | |
| | | NO: 24) | | GCTGCTAACAA | |
| AADC- | PRSETb | AADC- | 12 | TGTACGACGATGACGATAAGGATCCGAT | BamHI or |
| IKVAV | | IKVAV (SEQ | | GAACGCAAGTGAATTCCGAAGGAGAGG | EcoRI sites |
| (SEQ ID | | ID NO: 23) F | | GA | are |
| NO: 23) | | AADC- | 13 | AGCAGCCGGATCAAGCTTCGAATTCTTA | underlined. |
| | | IKVAV (SEQ | | CACGGCGACTTTGATTCCGCTGCCA | BamHI or |
| | | ID NO: 23) R | | | |
| AADC- | PTrcHisA | AADCF | 14 | TGACGATAAGGATCGATGGGGATCCATG | |
| IKVAV | | | | AACGCAAGTGAATTCCGAAGGAGAGGGA | |
| (SEQ ID | | AADC R | 15 | CTCCCTCTCTGCTCGCAGCACG | KpnI sites |
| NO: 23) | | DNA | 16 | ACGTGCTGCGAGCAGAGAGGGAGGGAG | are |
| template | | fragment of | | GAAGTGGCAGCTCTGGCGGCAGTGGAG | underlined. |
| | | IKVAV | | GGTCTGGTGGCAGCGGAATCAAAGTCG | |
| | | (SEQ ID NO: | | CCGTGTAGGGTACCATATGGGAATTCGA | |
| | | 23) | | AGCTTGGCTGT | |

TABLE 4
Protein coding sequences in this study.

| Name | SEQ ID NO | Protein coding sequence (CDS) or vector sequence | Note |
|---|---|---|---|
| RLuc8.6 | 17 | ATGCGGGGTTCTCATCATCATCATCATCATGGTATGGCTAGCATGACTGGT | His-tag |
| | | GGACAGCAAATGGGTCGGGATCTGTACGACGATGACGATAAGGATCCG | sequence is |
| | | ATGGCTTCCAAGGTGTACGACCCCGAGCAACGCAAACGCATGATCA | highlighted in |
| | | CTGGGCCTCAGTGGTGGGCTCGCTGCAAGCAAATGAACGTGCTGG | italics, and |
| | | ACTCCTTCATCAACTACTATGATTCCGAGAAGCACGCCGAGAACGC | RLuc8.6 in |
| | | CGTGATTTTTCTGCATGGTAACGCTACCTCCAGCTACCTGTGGAGG | bold. Other |
| | | CACGTCGTGCCTCACATCGAGCCCGTGGCTAGATGCATCATCCCTG | sequences are |
| | | ATCTGATCGGAATGGGTAAGTCCGGCAAGAGCGGGAATGGCTCAT | start codon, |
| | | ATCGCCTCCTGGATCACTACAAGTACCTCACCGCTTGGTTCGAGCT | T7 tag and X- |
| | | GCTGAACCTTCCAAAGAAAATCATCTTTGTGGGCCACGACTGGGGG | press tag on |
| | | AGCGCTCTGGCCTTTCACTACGCCTACGAGCACCAAGACAGGATCA | the original |
| | | AGGCCATCGTCCATATGGAGAGTGTCGTGGACGTGATCGAGTCCTG | vector |
| | | GATGGGGTGGCCTGACATCGAGGAGGAGCTGGCCCTGATCAAGAG | pRSETb. |
| | | CGAAGAGGGCGAGAAAATGGTGCTTGAGAATAACTTCTTCGTCGAG | |
| | | ACCCTGTTGCCAAGCAAGATCATGCGGAAACTGGAGCCTGAGGAGT | |
| | | TCGCTGCCTACCTGGAGCCATTCAAGGAGAAGGGCGAGGTTAGAC | |
| | | GGCCTACCCTCTCCTGGCCTCGCGAGATCCCTCTCGTTAAGGGAGG | |
| | | CAAGCCCGACGTCGTCCAGATTGTCCGCAACTACAACGCCTACCTT | |
| | | CGGGCCAGCGACGATCTGCCTAAGCTGTTCATCGAGTCCGACCCTG | |
| | | GGTTCTTTTCCAACGCTATTGTCGAGGGAGCTAAGAAGTTCCCTAA | |
| | | CACCGAGTTCGTGAAGGTGAAGGGCCTCCACTTCCTCCAGGAGGAC | |
| | | GCTCCAGATGAAATGGGTAAGTACATCAAGAGCTTCGTGGAGCGCG | |
| | | TGCTGAAGAACGAGCAG | |
| RLuc8. | 18 | ATGCGGGGTTCTCATCATCATCATCATCATGGTATGGCTAGCATGACTGGT | His-tag |
| 6- | | GGACAGCAAATGGGTCGGGATCTGTACGACGATGACGATAAGGATCCG | sequence is |
| IKVAV | | ATGGCTTCCAAGGTGTACGACCCCGAGCAACGCAAACGCATGATCA | highlighted in |
| (SEQ | | CTGGGCCTCAGTGGTGGGCTCGCTGCAAGCAAATGAACGTGCTGG | italics, |
| ID NO: | | ACTCCTTCATCAACTACTATGATTCCGAGAAGCACGCCGAGAACGC | RLuc8.6 in |
| 23) | | CGTGATTTTTCTGCATGGTAACGCTACCTCCAGCTACCTGTGGAGG | bold, GS |
| | | CACGTCGTGCCTCACATCGAGCCCGTGGCTAGATGCATCATCCCTG | linker in |
| | | ATCTGATCGGAATGGGTAAGTCCGGCAAGAGCGGGAATGGCTCAT | underlined, |
| | | ATCGCCTCCTGGATCACTACAAGTACCTCACCGCTTGGTTCGAGCT | and IKVAV |
| | | GCTGAACCTTCCAAAGAAAATCATCTTTGTGGGCCACGACTGGGGG | (SEQ ID NO: |
| | | AGCGCTCTGGCCTTTCACTACGCCTACGAGCACCAAGACAGGATCA | 23) in bold |
| | | AGGCCATCGTCCATATGGAGAGTGTCGTGGACGTGATCGAGTCCTG | and |
| | | GATGGGGTGGCCTGACATCGAGGAGGAGCTGGCCCTGATCAAGAG | underlined. |
| | | CGAAGAGGGCGAGAAAATGGTGCTTGAGAATAACTTCTTCGTCGAG | Other |
| | | ACCCTGTTGCCAAGCAAGATCATGCGGAAACTGGAGCCTGAGGAGT | sequences are |
| | | TCGCTGCCTACCTGGAGCCATTCAAGGAGAAGGGCGAGGTTAGAC | start codon, |
| | | GGCCTACCCTCTCCTGGCCTCGCGAGATCCCTCTCGTTAAGGGAGG | T7 tag and X- |
| | | CAAGCCCGACGTCGTCCAGATTGTCCGCAACTACAACGCCTACCTT | press tag on |
| | | CGGGCCAGCGACGATCTGCCTAAGCTGTTCATCGAGTCCGACCCTG | the original |
| | | GGTTCTTTTCCAACGCTATTGTCGAGGGAGCTAAGAAGTTCCCTAA | vector |
| | | CACCGAGTTCGTGAAGGTGAAGGGCCTCCACTTCCTCCAGGAGGAC | pRSETb. |
| | | GCTCCAGATGAAATGGGTAAGTACATCAAGAGCTTCGTGGAGCGCG | |
| | | TGCTGAAGAACGAGCAGGGAGGAAGTGGCAGCTCTGGCGGCAGTGGA | |
| | | GGGTCTGGTGGCAGCGGAATCAAAGTCGCCGTG | |
| RLuc8. | 19 | ATGCGGGGTTCTCATCATCATCATCATCATGGTATGGCTAGCATGACTGGT | His-tag |
| 6- | | GGACAGCAAATGGGTCGGGATCTGTACGACGATGACGATAAGGATCCG | sequence is |
| YIGSR | | ATGGCTTCCAAGGTGTACGACCCCGAGCAACGCAAACGCATGATCA | highlighted in |
| (SEQ | | CTGGGCCTCAGTGGTGGGCTCGCTGCAAGCAAATGAACGTGCTGG | italics, |
| ID | | ACTCCTTCATCAACTACTATGATTCCGAGAAGCACGCCGAGAACGC | RLuc8.6 in |
| NO: | | CGTGATTTTTCTGCATGGTAACGCTACCTCCAGCTACCTGTGGAGG | bold, GS |
| 25) | | CACGTCGTGCCTCACATCGAGCCCGTGGCTAGATGCATCATCCCTG | linker in |
| | | ATCTGATCGGAATGGGTAAGTCCGGCAAGAGCGGGAATGGCTCAT | underlined, |
| | | ATCGCCTCCTGGATCACTACAAGTACCTCACCGCTTGGTTCGAGCT | and YIGSR |
| | | GCTGAACCTTCCAAAGAAAATCATCTTTGTGGGCCACGACTGGGGG | (SEQ ID NO: |
| | | AGCGCTCTGGCCTTTCACTACGCCTACGAGCACCAAGACAGGATCA | 25) in bold |
| | | AGGCCATCGTCCATATGGAGAGTGTCGTGGACGTGATCGAGTCCTG | and |
| | | GATGGGGTGGCCTGACATCGAGGAGGAGCTGGCCCTGATCAAGAG | underlined. |
| | | CGAAGAGGGCGAGAAAATGGTGCTTGAGAATAACTTCTTCGTCGAG | Other |
| | | ACCCTGTTGCCAAGCAAGATCATGCGGAAACTGGAGCCTGAGGAGT | sequences are |
| | | TCGCTGCCTACCTGGAGCCATTCAAGGAGAAGGGCGAGGTTAGAC | start codon, |
| | | GGCCTACCCTCTCCTGGCCTCGCGAGATCCCTCTCGTTAAGGGAGG | T7 tag and X- |
| | | CAAGCCCGACGTCGTCCAGATTGTCCGCAACTACAACGCCTACCTT | press tag on |
| | | CGGGCCAGCGACGATCTGCCTAAGCTGTTCATCGAGTCCGACCCTG | the original |
| | | GGTTCTTTTCCAACGCTATTGTCGAGGGAGCTAAGAAGTTCCCTAA | vector |
| | | CACCGAGTTCGTGAAGGTGAAGGGCCTCCACTTCCTCCAGGAGGAC | pRSETb. |
| | | GCTCCAGATGAAATGGGTAAGTACATCAAGAGCTTCGTGGAGCGCG | |
| | | TGCTGAAGAACGAGCAGGGAGGAAGTGGCAGCTCTGGCGGCAGTGGA | |
| | | GGGTCTGGTGGCAGCGGATACATTGGTTCCAGA | |
| RLuc8. | 20 | ATGCGGGGTTCTCATCATCATCATCATCATGGTATGGCTAGCATGACTGGT | His-tag |
| 6- | | GGACAGCAAATGGGTCGGGATCTGTACGACGATGACGATAAGGATCCG | sequence is |
| GRGDS | | ATGGCTTCCAAGGTGTACGACCCCGAGCAACGCAAACGCATGATCA | highlighted in |
| (SEQ | | CTGGGCCTCAGTGGTGGGCTCGCTGCAAGCAAATGAACGTGCTGG | italics, |
| ID | | ACTCCTTCATCAACTACTATGATTCCGAGAAGCACGCCGAGAACGC | RLuc8.6 in |
| NO: | | CGTGATTTTTCTGCATGGTAACGCTACCTCCAGCTACCTGTGGAGG | bold, GS |
| 24) | | CACGTCGTGCCTCACATCGAGCCCGTGGCTAGATGCATCATCCCTG | linker in |
| | | ATCTGATCGGAATGGGTAAGTCCGGCAAGAGCGGGAATGGCTCAT | underlined, |
| | | ATCGCCTCCTGGATCACTACAAGTACCTCACCGCTTGGTTCGAGCT | and GRGDS |
| | | GCTGAACCTTCCAAAGAAAATCATCTTTGTGGGCCACGACTGGGGG | (SEQ ID NO: |
| | | AGCGCTCTGGCCTTTCACTACGCCTACGAGCACCAAGACAGGATCA | 24) in bold |
| | | AGGCCATCGTCCATATGGAGAGTGTCGTGGACGTGATCGAGTCCTG | and |
| | | GATGGGGTGGCCTGACATCGAGGAGGAGCTGGCCCTGATCAAGAG | underlined. |
| | | CGAAGAGGGCGAGAAAATGGTGCTTGAGAATAACTTCTTCGTCGAG | Other |
| | | ACCCTGTTGCCAAGCAAGATCATGCGGAAACTGGAGCCTGAGGAGT | sequences are |
| | | TCGCTGCCTACCTGGAGCCATTCAAGGAGAAGGGCGAGGTTAGAC | start codon, |
| | | GGCCTACCCTCTCCTGGCCTCGCGAGATCCCTCTCGTTAAGGGAGG | T7 tag and X- |
| | | CAAGCCCGACGTCGTCCAGATTGTCCGCAACTACAACGCCTACCTT | press tag on |
| | | CGGGCCAGCGACGATCTGCCTAAGCTGTTCATCGAGTCCGACCCTG | the original |
| | | GGTTCTTTTCCAACGCTATTGTCGAGGGAGCTAAGAAGTTCCCTAA | vector |
| | | CACCGAGTTCGTGAAGGTGAAGGGCCTCCACTTCCTCCAGGAGGAC | pRSETb. |
| | | GCTCCAGATGAAATGGGTAAGTACATCAAGAGCTTCGTGGAGCGCG | |
| | | TGCTGAAGAACGAGCAGGGAGGAAGTGGCAGCTCTGGCGGCAGTGGA | |
| | | GGGTCTGGTGGCAGCGGAGGTCGTGGTGATAGT | |
| AADC- | 21 | ATGCGGGGTTCTCATCATCATCATCATCATGGTATGGCTAGCATGACTGGT | His-tag |
| IKVAV | | GGACAGCAAATGGGTCGGGATCTGTACGACGATGACGATAAGGATCCG | sequence is |
| (SEQ | | ATGAACGCAAGTGAATTCCGAAGGAGAGGGAAGGAGATGGTGGAT | highlighted in |
| ID NO: | | TACATGGCCAACTACATGGAAGGCATTGAGGGACGCCAGGTCTACC | italics, |
| 23) | | CTGACGTGGAGCCCGGGTACCTGCGGCCGCTGATCCCTGCCGCTG | AADC in |
| | | CCCCTCAGGAGCCAGACACGTTTGAGGACATCATCAACGACGTTGA | bold, GS |
| | | GAAGATAATCATGCCTGGGGTGACGCACTGGCACAGCCCCTACTTC | linker in |
| | | TTCGCCTACTTCCCCACTGCCAGCTCGTACCCGGCCATGCTTGCGG | underlined, |
| | | ACATGCTGTGCGGGGCCATTGGCTGCATCGGCTTCTCCTGGGCGGC | and |
| | | AAGCCCAGCATGCACAGAGCTGGAGACTGTGATGATGGACTGGCT | IKVAV(SEQ |
| | | CGGGAAGATGCTGGAACTACCAAAGGCATTTTTGAATGAGAAAGCT | ID NO: 23) in |
| | | GGAGAAGGGGGAGGAGTGATCCAGGGAAGTGCCAGTGAAGCCACC | bold and |
| | | CTGGTGGCCCTGCTGGCCGCTCGGACCAAAGTGATCCATCGGCTGC | underlined. |
| | | AGGCAGCGTCCCCAGAGCTCACACAGGCCGCTATCATGGAGAAGC | Other |
| | | TGGTGGCTTACTCATCCGATCAGGCACACTCCTCAGTGGAAAGAGC | sequences are |
| | | TGGGTTAATTGGTGGAGTGAAATTAAAAGCCATCCCCTCAGATGGC | start codon, |
| | | AACTTCGCCATGCGTGCGTCTGCCCTGCAGGAAGCCCTGGAGAGA | T7 tag and X- |
| | | GACAAAGCGGCTGGCCTGATTCCTTTCTTTATGGTTGCCACCCTGG | press tag on |
| | | GGACCACAACATGCTGCTCCTTTGACAATCTCTTAGAAGTCGGTCC | the original |
| | | TATCTGCAACAAGGAAGACATATGGCTGCACGTTGATGCAGCCTAC | vector |
| | | GCAGGCAGTGCATTCATCTGCCCTGAGTTCCGGCACCTTCTGAATG | pRSETb. |
| | | GAGTGGAGTTTGCAGATTCATTCAACTTTAATCCCCACAAATGGCT | |
| | | ATTGGTGAATTTTGACTGTTCTGCCATGTGGGTGAAAAAGAGAACA | |
| | | GACTTAACGGGAGCCTTTAGACTGGACCCCACTTACCTGAAGCACA | |
| | | GCCATCAGGATTCAGGGCTTATCACTGACTACCGGCATTGGCAGAT | |
| | | ACCACTGGGCAGAAGATTTCGCTCTTTGAAAATGTGGTTTGTATTT | |
| | | AGGATGTATGGAGTCAAAGGACTGCAGGCTTATATCCGCAAGCATG | |
| | | TCCAGCTGTCCCATGAGTTTGAGTCACTGGTGCGCCAGGATCCCCG | |
| | | CTTTGAAATCTGTGTGGAAGTCATTCTGGGGCTTGTCTGCTTTCGG | |
| | | CTAAAGGGTTCCAACAAAGTGAATGAAGCTCTTCTGCAAAGAATAA | |
| | | ACAGTGCCAAAAAAATCCACTTGGTTCCATGTCACCTCAGGGACAA | |
| | | GTTTGTCCTGCGCTTTGCCATCTGTTCTCGCACGGTGGAATCTGCC | |
| | | CATGTGCAGCGGGCCTGGGAACACATCAAAGAGCTGGCGGCCGAC | |
| | | GTGCTGCGAGCAGAGAGGGAGGGAGGAAGTGGCAGCTCTGGCGGCA | |
| | | GTGGAGGGTCTGGTGGCAGCGGAATCAAAGTCGCCGTG | |
| pRSET | 22 | TTCATTTTTAATTTAAAAGGATCTAGGTGAAGATCCTTTTTGATAATCTC | |
| b vector | | ATGACCAAAATCCCTTAACGTGAGTTTTCGTTCCACTGAGCGTCAGACC | |
| | | CCGTAGAAAAGATCAAAGGATCTTCTTGAGATCCTTTTTTTCTGCGCGT | |
| | | AATCTGCTGCTTGCAAACAAAAAAACCACCGCTACCAGCGGTGGTTTGT | |
| | | TTGCCGGATCAAGAGCTACCAACTCTTTTTCCGAAGGTAACTGGCTTCA | |
| | | GCAGAGCGCAGATACCAAATACTGTTCTTCTAGTGTAGCCGTAGTTAGG | |
| | | CCACCACTTCAAGAACTCTGTAGCACCGCCTACATACCTCGCTCTGCTA | |
| | | ATCCTGTTACCAGTGGCTGCTGCCAGTGGCGATAAGTCGTGTCTTACCG | |
| | | GGTTGGACTCAAGACGATAGTTACCGGATAAGGCGCAGCGGTCGGGCT | |
| | | GAACGGGGGGTTCGTGCACACAGCCCAGCTTGGAGCGAACGACCTACA | |
| | | CCGAACTGAGATACCTACAGCGTGAGCTATGAGAAAGCGCCACGCTTC | |
| | | CCGAAGGGAGAAAGGCGGACAGGTATCCGGTAAGCGGCAGGGTCGGA | |
| | | ACAGGAGAGCGCACGAGGGAGCTTCCAGGGGGAAACGCCTGGTATCTT | |
| | | TATAGTCCTGTCGGGTTTCGCCACCTCTGACTTGAGCGTCGATTTTTGTG | |
| | | ATGCTCGTCAGGGGGGCGGAGCCTATGGAAAAACGCCAGCAACGCGGC | |
| | | CTTTTTACGGTTCCTGGCCTTTTGCTGGCCTTTTGCTCACATGTTCTTTCC | |
| | | TGCGTTATCCCCTGATTCTGTGGATAACCGTATTACCGCCTTTGAGTGAG | |
| | | CTGATACCGCTCGCCGCAGCCGAACGACCGAGCGCAGCGAGTCAGTGA | |
| | | GCGAGGAAGCGGAAGAGCGCCCAATACGCAAACCGCCTCTCCCCGCGC | |
| | | GTTGGCCGATTCATTAATGCAGGATCTCGATCCCGCGAAATTAATACGA | |
| | | CTCACTATAGGGAGACCACAACGGTTTCCCTCTAGAAATAATTTTGTTT | |
| | | AACTTTAAGAAGGAGATATACATATGCGGGGTTCTCATCATCATCATCA | |
| | | TCATGGTATGGCTAGCATGACTGGTGGACAGCAAATGGGTCGGGATCT | |
| | | GTACGACGATGACGATAAGGATCCGATGGTGAGCAAGGGCGAGGAGGT | |
| | | CATCAAAGAGTTCATGCGCTTCAAGGTGCGCATGGAGGGCTCCATGAA | |
| | | CGGCCACGAGTTCGAGATCGAGGGCGAGGGCGAGGGCCGCCCCTACGA | |
| | | GGGCACCCAGACCGCCAAGCTGAAGGTGACCAAGGGCGGCCCCCTGCC | |
| | | CTTCGCCTGGGACATCCTGTCCCCCCAGTTCATGTACGGCTCCAAGGCG | |
| | | TACGTGAAGCACCCCGCCGACATCCCCGATTACAAGAAGCTGTCCTTCC | |
| | | CCGAGGGCTTCAAGTGGGAGCGCGTGATGAACTTCGAGGACGGCGGTC | |
| | | TGGTGACCGTGACCCAGGACTCCTCCCTGCAGGACGGCACGCTGATCTA | |
| | | CAAGGTGAAGATGCGCGGCACCAACTTCCCCCCCGACGGCCCCGTAAT | |
| | | GCAGAAGAAGACCATGGGCTGGGAGGCCTCCACCGAGCGCCTGTACCC | |
| | | CCGCGACGGCGTGCTGAAGGGCGAGATCCACCAGGCCCTGAAGCTGAA | |
| | | GGACGGCGGCCACTACCTGGTGGAGTTCAAGACCATCTACATGGCCAA | |
| | | GAAGCCCGTGCAACTGCCCGGCTACTACTACGTGGACACCAAGCTGGA | |
| | | CATCACCTCCCACAACGAGGACTACACCATCGTGGAACAGTACGAGCG | |
| | | CTCCGAGGGCCGCCACCACCTGTTCCTGGGGCATGGCACCGGCAGCACC | |
| | | GGCAGCGGCAGCTCCGGCACCGCCTCCTCCGAGGACAACAACATGGCC | |
| | | GTCATCAAAGAGTTCATGCGCTTCAAGGTGCGCATGGAGGGCTCCATGA | |
| | | ACGGCCACGAGTTCGAGATCGAGGGCGAGGGCGAGGGCCGCCCCTACG | |
| | | AGGGCACCCAGACCGCCAAGCTGAAGGTGACCAAGGGCGGCCCCCTGC | |
| | | CCTTCGCCTGGGACATCCTGTCCCCCCAGTTCATGTACGGCTCCAAGGC | |
| | | GTACGTGAAGCACCCCGCCGACATCCCCGATTACAAGAAGCTGTCCTTC | |
| | | CCCGAGGGCTTCAAGTGGGAGCGCGTGATGAACTTCGAGGACGGCGGT | |
| | | CTGGTGACCGTGACCCAGGACTCCTCCCTGCAGGACGGCACGCTGATCT | |
| | | ACAAGGTGAAGATGCGCGGCACCAACTTCCCCCCCGACGGCCCCGTAA | |
| | | TGCAGAAGAAGACCATGGGCTGGGAGGCCTCCACCGAGCGCCTGTACC | |
| | | CCCGCGACGGCGTGCTGAAGGGCGAGATCCACCAGGCCCTGAAGCTGA | |
| | | AGGACGGCGGCCACTACCTGGTGGAGTTCAAGACCATCTACATGGCCA | |
| | | AGAAGCCCGTGCAACTGCCCGGCTACTACTACGTGGACACCAAGCTGG | |
| | | ACATCACCTCCCACAACGAGGACTACACCATCGTGGAACAGTACGAGC | |
| | | GCTCCGAGGGCCGCCACCACCTGTTCCGGCTGGAAGATTTCGTTGGGGA | |
| | | CTGGCGACAGACAGCCGGCTACAACCTGGACCAAGTCCTTGAACAGGG | |
| | | AGGTGTGTCCAGTTTGTTTCAGAATCTCGGGGTGTCCGTAACTCCGATC | |
| | | CAAAGGATTGTCCTGAGCGGTGAAAATGGGCTGAAGATCGACATCCAT | |
| | | GTCATCATCCCGTATGAAGGTCTGAGCGGCGACCAAATGGGCCAGATC | |
| | | GAAAAAATTTTTAAGGTGGTGTACCCTGTGGATGATCATCACTTTAAGG | |
| | | TGATCCTGCACTATGGCACACTGGTAATCGACGGGGTTACGCCGAACAT | |
| | | GATCGACTATTTCGGACGGCCGTATGAAGGCATCGCCGTGTTCGACGGC | |
| | | AAAAAGATCACTGTAACAGGGACCCTGTGGAACGGCAACAAAATTATC | |
| | | GACGAGCGCCTGATCAACCCCGACGGCTCCCTGCTGTTCCGAGTAACCA | |
| | | TCAACGGAGTGACCGGCTGGCGGCTGTGCGAACGCATTCTGGCGTAAG | |
| | | AATTCGAAGCTTGATCCGGCTGCTAACAAAGCCCGAAAGGAAGCTGAG | |
| | | TTGGCTGCTGCCACCGCTGAGCAATAACTAGCATAACCCCTTGGGGCCT | |
| | | CTAAACGGGTCTTGAGGGGTTTTTTGCTGAAAGGAGGAACTATATCCGG | |
| | | ATCTGGCGTAATAGCGAAGAGGCCCGCACCGATCGCCCTTCCCAACAGT | |
| | | TGCGCAGCCTGAATGGCGAATGGGACGCGCCCTGTAGCGGCGCATTAA | |
| | | GCGCGGCGGGTGTGGTGGTTACGCGCAGCGTGACCGCTACACTTGCCA | |
| | | GCGCCCTAGCGCCCGCTCCTTTCGCTTTCTTCCCTTCCTTTCTCGCCACG | |
| | | TTCGCCGGCTTTCCCCGTCAAGCTCTAAATCGGGGGCTCCCTTTAGGGTT | |
| | | CCGATTTAGTGCTTTACGGCACCTCGACCCCAAAAAACTTGATTAGGGT | |
| | | GATGGTTCACGTAGTGGGCCATCGCCCTGATAGACGGTTTTTCGCCCTT | |
| | | TGACGTTGGAGTCCACGTTCTTTAATAGTGGACTCTTGTTCCAAACTGG | |
| | | AACAACACTCAACCCTATCTCGGTCTATTCTTTTGATTTATAAGGGATTT | |
| | | TGCCGATTTCGGCCTATTGGTTAAAAAATGAGCTGATTTAACAAAAATT | |
| | | TAACGCGAATTTTAACAAAATATTAACGCTTACAATTTAGGTGGCACTT | |
| | | TTCGGGGAAATGTGCGCGGAACCCCTATTTGTTTATTTTTCTAAATACAT | |
| | | TCAAATATGTATCCGCTCATGAGACAATAACCCTGATAAATGCTTCAAT | |
| | | AATATTGAAAAAGGAAGAGTATGAGTATTCAACATTTCCGTGTCGCCCT | |
| | | TATTCCCTTTTTTGCGGCATTTTGCCTTCCTGTTTTTGCTCACCCAGAAAC | |
| | | GCTGGTGAAAGTAAAAGATGCTGAAGATCAGTTGGGTGCACGAGTGGG | |
| | | TTACATCGAACTGGATCTCAACAGCGGTAAGATCCTTGAGAGTTTTCGC | |
| | | CCCGAAGAACGTTTTCCAATGATGAGCACTTTTAAAGTTCTGCTATGTG | |
| | | GCGCGGTATTATCCCGTATTGACGCCGGGCAAGAGCAACTCGGTCGCCG | |
| | | CATACACTATTCTCAGAATGACTTGGTTGAGTACTCACCAGTCACAGAA | |
| | | AAGCATCTTACGGATGGCATGACAGTAAGAGAATTATGCAGTGCTGCC | |
| | | ATAACCATGAGTGATAACACTGCGGCCAACTTACTTCTGACAACGATCG | |
| | | GAGGACCGAAGGAGCTAACCGCTTTTTTGCACAACATGGGGGATCATG | |
| | | TAACTCGCCTTGATCGTTGGGAACCGGAGCTGAATGAAGCCATACCAA | |
| | | ACGACGAGCGTGACACCACGATGCCTGTAGCAATGGCAACAACGTTGC | |
| | | GCAAACTATTAACTGGCGAACTACTTACTCTAGCTTCCCGGCAACAATT | |
| | | AATAGACTGGATGGAGGCGGATAAAGTTGCAGGACCACTTCTGCGCTC | |
| | | GGCCCTTCCGGCTGGCTGGTTTATTGCTGATAAATCTGGAGCCGGTGAG | |
| | | CGTGGGTCTCGCGGTATCATTGCAGCACTGGGGCCAGATGGTAAGCCCT | |
| | | CCCGTATCGTAGTTATCTACACGACGGGGAGTCAGGCAACTATGGATGA | |
| | | ACGAAATAGACAGATCGCTGAGATAGGTGCCTCACTGATTAAGCATTG | |
| | | GTAACTGTCAGACCAAGTTTACTCATATATACTTTAGATTGATTTAAAAC | |

TABLE 5
FUS target coordinates used for this study.

| | | | Medio- | Anterior- | Dorso- |
|---|---|---|---|---|---|
| Related | | FUS | lateral | posterior | ventral |
| FIG. | Experiment | target | (mm) | (mm) | (mm) |
| | IHC | Site-1 | −2.74 | −0.1 | 4.33 |
| FIG. | analysis of | Site-2 | −1.85 | −0.1 | 4 |
| 2b-c | RLuc8.6 | | | | |
| | (4 sites) | Site-3 | −2.5 | 0.13 | 4.48 |
| | | Site-4 | −1.6 | 0.13 | 3.95 |
| FIG. | In vivo BLI | Site-1 | −2.42 | −0.12 | 4.3 |
| 2d-e | (1 site) | | | | |
| | Ex vivo BLI | Site-1 | −2.42 | −0.45 | 3.43 |
| FIG. 5 | (3 sites) | Site-2 | −1.3 | −1.57 | 2.88 |
| | | Site-3 | −1.22 | −3.8 | 2.88 |
| FIG. 7 | c-Fos | Site-1 | −2.42 | −0.12 | 4.1 |
| | activation | | | | |
| | (1 site) | | | | |
| FIG. 11 | Behavior | Site-1 | −2.32 | 0.18 | 4 |
| | test (2 sites) | Site-2 | −2.92 | −0.62 | 4 |

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method for applying site-specific brain therapeutics, comprising:
delivering an enzyme to a selected site of a brain; and
converting, via the enzyme, an inactive prodrug into an active drug at the selected site of the brain.

2. The method of claim 1, wherein the enzyme is delivered non-invasively.

3. The method of claim 1, wherein the enzyme comprises an engineered protein enzyme.

4. The method of claim 1, wherein the enzyme comprises a catalytic enzyme.

5. The method of claim 1, wherein the inactive prodrug is blood-brain-barrier permeable prodrug.

6. The method of claim 1, wherein the enzyme is delivered to the brain using focused ultrasound blood brain barrier opening (FUS-BBBO).

7. The method of claim 1, wherein the active drug is a neuroactive drug.

8. The method of claim 1, wherein delivering the enzyme comprises the enzyme binding within an interstitial space of the brain.

9. The method of claim 1, wherein delivering the enzyme comprises encoding directly into one or more cells via gene delivery.

10. The method of claim 9, wherein the gene delivery comprises noninvasively delivering AADC to the brain using focused ultrasound blood brain barrier opening (FUS-BBBO).

11. The method of claim 1, wherein delivering the enzyme comprises the enzyme binding to the brain parenchyma.

12. The method of claim 1, further comprising:
modulating neuronal activity around the enzyme by supplying inactive prodrug over time for conversion to the active drug.

13. The method of claim 12, wherein the modulated neuronal activity is spatially-specific within the brain.

14. The method of claim 12, wherein the inactive prodrug is supplied systematically over time.

15. The method of claim 14, wherein the inactive prodrug is supplied systematically over time through an intact blood-brain-barrier.

16. The method of claim 1, wherein the enzyme is present at the selected site of the brain for one or more days.

17. The method of claim 1, wherein the conversion of the inactive prodrug into the active drug at the selected site of the brain by the enzyme is tuned by controlling the varying a dose of the prodrug.

18. An engineered enzyme comprising:
an aromatic-L-amino-acid decarboxylase (AADC); and
an extracellular matrix (ECM)-mimicking peptide fused to the AADC.

19. The engineered enzyme of claim 18, wherein the ECM-mimicking peptide comprises IKVAV (SEQ ID NO: 23), GRGDS (SEQ ID NO: 24), or YIGSR (SEQ ID NO: 25).

20. The engineered enzyme of claim 18, wherein the AADC converts L-DOPA to dopamine, wherein the L-DOPA is capable of passing through an intact blood-brain-barrier.

21. A method for modulating neuronal activity at a spatially specific site within a brain, comprising:
administering an inert prodrug to a subject, wherein the inert prodrug is capable of penetrating an intact blood-brain-barrier and wherein the inert prodrug is converted to an active drug at the spatially specific site within the brain; and
varying a dose of the inert prodrug over time to achieve a corresponding or modulated dose of the active drug at the spatially specific site.

22. The method of claim 21, wherein the inert prodrug is converted to the active drug by an engineered enzyme present at the spatially specific site.
